(19) United States
(12) Patent Application Publication
(10) Pub. No.: US 2025/0021003 A1
(43) Pub. Date: Jan. 16, 2025

(54) POLYMER, MONOMER, RESIST COMPOSITION COMPRISING THE SAME AND METHOD OF FORMING PATTERN USING THE SAME

(71) Applicant: Samsung Electronics Co., Ltd., Suwon-si (KR)

(72) Inventors: Cheol KANG, Suwon-si (KR); Haengdeog KOH, Suwon-si (KR); Yoonhyun KWAK, Suwon-si (KR); Minsang KIM, Suwon-si (KR); Beomseok KIM, Suwon-si (KR); Hana KIM, Suwon-si (KR); Hoyoon PARK, Suwon-si (KR); Chanjae AHN, Suwon-si (KR); Jaejun LEE, Suwon-si (KR); Sungwon CHOI, Suwon-si (KR)

(73) Assignee: Samsung Electronics Co., Ltd., Suwon-si (KR)

(21) Appl. No.: 18/537,601

(22) Filed: Dec. 12, 2023

(30) Foreign Application Priority Data
Jun. 27, 2023 (KR) 10-2023-0082888

(51) Int. Cl. G03F 7/039; G03F 7/004; G03F 7/038

(52) U.S. Cl. CPC G03F 7/0397; G03F 7/0048; G03F 7/0382

(57) ABSTRACT

Provided are a polymer including a first repeating unit represented by Formula 1 below, a resist composition including the same, a method of forming a pattern by using the same, and a monomer represented by Formula 10 below.

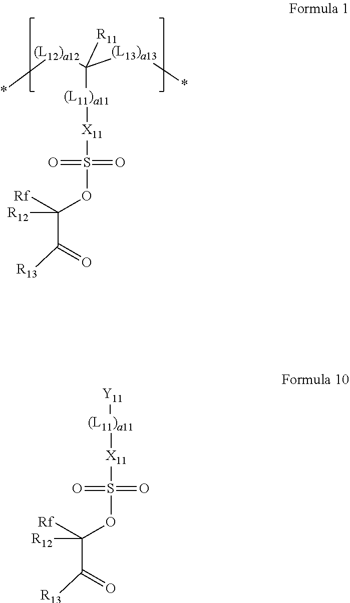

In Formulae 1 and 10, L₁₁ to L₁₃, a11 to a13, X₁₁, Rf, and R₁₁ to R₁₃ are as described in the specification.

### CROSS-REFERENCE TO RELATED APPLICATION

This application is based on and claims priority under 35 U.S.C. § 119 to Korean Patent Application No. 10-2023-0082888, filed on Jun. 27, 2023 in the Korean Intellectual Property Office, the disclosure of which is incorporated by reference herein in its entirety.

### BACKGROUND

### 1. Field

The disclosure relates to a polymer, a monomer, a resist composition including the same, and a method of forming a pattern by using the same.

### 2. Description of the Related Art

In semiconductor manufacturing, photoresists of which physical properties change in response to light are used to form fine patterns. Among them, chemically amplified photoresists have been widely used. A chemically amplified photoresist enables patterning because a base resin of the chemically amplified photoresist reacts with an acid produced by a reaction between light and a photoacid generator, resulting in a change in solubility of the base resin in a developer.

However, in the case of the chemically amplified photoresist, the produced acid may diffuse to an unexposed region, causing problems such as a decrease in uniformity of a pattern or an increase in surface roughness. In addition, as semiconductor processes become increasingly finer, it is not easy to control diffusion of an acid, and thus there may be a need to develop a new resist method.

Recently, in order to overcome the limits of chemically amplified photoresists, attempts have been made to develop materials of which physical properties change with exposure to light. However, a dose required for light exposure is still high.

Therefore, there may be a need to develop a material of which physical properties change at a low dose via a quick reaction.

### SUMMARY

Therefore, provided are a polymer, a monomer, a resist composition including the same and a method of forming a pattern using the same, those physical properties, particularly solubility are changed by exposure light even at a low dose of light.

Additional aspects will be set forth in part in the description which follows and, in part, will be apparent from the description, or may be learned by practice of the presented embodiments of the disclosure.

According to an embodiment, a polymer may include a first repeating unit represented by Formula 1 below.

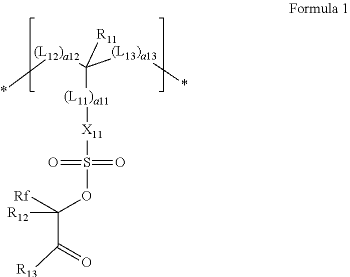

In Formula 1,
L₁₁ to L₁₃ each independently may be a single bond; O; S; C(═O); C(═O)O; OC(═O); C(═O)NH; NHC(═O); or a linear, branched, or cyclic C₁-C₃₀ divalent hydrocarbon group optionally including a heteroatom,
a11 to a13 each independently may be an integer from 1 to 4,
X₁₁ may be a linear, branched, or cyclic C₁-C₃₀ divalent hydrocarbon group optionally including a heteroatom other than F,
Rf may be a linear, branched, or cyclic C₁-C₃₀ monovalent hydrocarbon group including at least one F and optionally including a heteroatom other than F,
R₁₁ and R₁₂ each independently may be hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
R₁₃ may be a substituted or unsubstituted C₆-C₃₀ aryl group or a substituted or unsubstituted C₁-C₃₀ heteroaryl group,
an adjacent two among Rf, R₁₂, and R₁₃ optionally may bind to each other to form a ring, and
* may be a binding site with an adjacent atom.

According to an embodiment, a monomer may be represented by Formula 10 below.

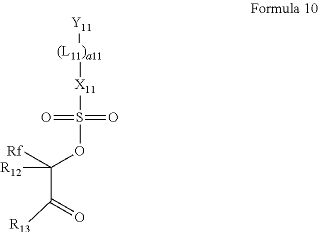

In Formula 10,
Y₁₁ may be a polymerizable group;
L₁₁ may be a single bond; O; S; C(═O); C(═O)O; OC(═O); C(═O)NH; NHC(═O); or a linear, branched, or cyclic C₁-C₃₀ divalent hydrocarbon group optionally including a heteroatom,
a11 may be an integer from 1 to 4,
X₁₁ may be a linear, branched, or cyclic C₁-C₃₀ divalent hydrocarbon group optionally including a heteroatom other than F,
Rf may be a linear, branched, or cyclic C₁-C₃₀ monovalent hydrocarbon group including at least one F and optionally including a heteroatom other than F,
R₁₂ may be hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
R₁₃ may be a substituted or unsubstituted C₆-C₃₀ aryl group or a substituted or unsubstituted C₁-C₃₀ heteroaryl group,
an adjacent two among Rf, R₁₂, and R₁₃, optionally may bind to each other to form a ring.

According to an embodiment, a resist composition may include the above-described polymer and an organic solvent.

According to an embodiment, a method of forming a pattern may include forming a resist film by applying the above-described resist composition, exposing at least one portion of the resist film to high-energy rays to provide an exposed resist film, and developing the exposed resist film using a developer.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a flowchart illustrating a method of forming a pattern, according to an embodiment.

FIGS. 2A to 2C are side cross-sectional views showing a method of forming a pattern, according to an embodiment.

FIGS. 3A to 3F are graphs showing thickness changes of films after developing Polymers P1 to P6 using DUV rays at various doses.

FIGS. 4A to 4E are side cross-sectional views illustrating a method of forming a patterned structure according to an embodiment.

FIGS. 5A to 5E are side cross-sectional views illustrating a method of forming a semiconductor device according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., +10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified as “about” or “substantially,” it will be understood that these values and shapes should be construed as including a manufacturing or operational tolerance (e.g., +10%) around the stated numerical values or shapes. When ranges are specified, the range includes all values therebetween such as increments of 0.1%.

As the disclosure below allows for various changes and numerous embodiments, particular embodiments will be illustrated in the drawings and described in detail in the written description. However, this is not intended to limit the disclosure to particular modes of practice, and it is to be appreciated that all modifications, equivalents, and substitutes that do not depart from the spirit and technical scope of the disclosure are encompassed in the disclosure. In the following description of the disclosure, a detailed description of known functions and configurations incorporated herein will be omitted when it may obscure the subject matter of the disclosure.

Although the terms “first”, “second”, “third”, and the like may be used herein to describe various elements, these terms are only used to distinguish one element from another and the order, type, or the like of the elements are not limited thereby.

Throughout the specification, it will be understood that when one element such as layer, film, region, or plate, is referred to as being “on” another element, it may be directly on the other element or intervening elements may also be present therebetween.

An expression used in the singular encompasses the expression of the plural, unless it has a clearly different meaning in the context. In the present specification, it is to be understood that the terms such as “including” or “having,” etc., are intended to indicate the existence of the features, numbers, operations, elements, parts, components, materials, or combinations thereof disclosed in the specification, and are not intended to preclude the possibility that one or more other features, numbers, operations, elements, parts, components, materials, or combinations thereof may exist or may be added, unless otherwise stated.

Whenever a range of values is recited, the range includes all values corresponding to the range as clearly recited and also includes boundaries of the range. Therefore, a range “X to Y” includes all numbers between X and Y and also includes X and Y.

As used herein, the term “Cₓ-C_{y}” means that the number of carbon atoms constituting a substituent is from x to y. For example, the term “C₁-C₆” means that the number of carbon atoms constituting a substituent is from 1 to 6, and the term “C₆-C₂₀” means that the number of carbon atoms constituting a substituent is from 6 to 20.

As used herein, the term “monovalent hydrocarbon group” refers to a monovalent moiety derived from an organic compound including carbon and hydrogen or derivatives thereof, and examples thereof may include a linear or branched alkyl group (e.g. a methyl group, an ethyl group, a propyl group, an isopropyl group, a butyl group, an isobutyl group, a sec-butyl group, a tert-butyl group, a pentyl group, a neopentyl group, a hexyl group, a heptyl group, a 2-ethylhexyl group, and a nonyl group); a monovalent saturated cycloaliphatic hydrocarbon group (a cycloalkyl group, e.g., a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a 1-adamantyl group, a 2-adamantyl group, a 1-adamantylmethyl group, a norbornyl group, a norbornylmethyl group, a tricyclodecanyl group, a tetracyclododecanyl group, a tetracyclododecanylmethyl group, and a dicyclohexylmethyl group); a monovalent unsaturated aliphatic hydrocarbon group (an alkenyl group or an alkynyl group, e.g., an allyl group); a monovalent unsaturated cycloaliphatic hydrocarbon group (cycloalkenyl group, e.g., a 3-cyclohexenyl group); an aryl group (e.g., a phenyl group, a 1-naphthyl group, and a 2-naphthyl group); an arylalkyl group (e.g., a benzyl group and a diphenylmethyl group); a heteroatom-containing monovalent hydrocarbon group (e.g., a tetrahydrofuranyl group, a methoxymethyl group, an ethoxymethyl group, a methylthiomethyl group, an aceteamidemethyl group, a trifluoroethyl group, a 2-(methoxyethoxy)methyl group, an acetoxymethyl group, a 2-carboxyl-1-cyclohexyl group, a 2-oxopropyl group, a 4-oxo-1-adamantyl group, and a 3-oxocyclohexyl group), or any combination thereof. Also, because, in these groups, some hydrogen atoms may be substituted with a moiety including a heteroatom, such as oxygen, sulfur, nitrogen, or a halogen atom, or some carbon atoms may be substituted with a moiety including a heteroatom, such as oxygen, sulfur, or nitrogen, these groups may include a hydroxyl group, a cyano group, a carbonyl group, a carboxyl group, an ether bond, an ester bond, a sulfonate ester bond, a carbonate, a lactone ring, a sultone ring, a carboxylic anhydride moiety, or a haloalkyl moiety.

As used herein, the term “divalent hydrocarbon group” refers to a divalent moiety prepared by substituting at least one hydrogen atom of the monovalent hydrocarbon group with a binding site with an adjacent atom. The divalent hydrocarbon group may include, for example, a linear or branched alkylene group, a cycloalkylene group, an alkenylene group, an alkynylene group, a cycloalkylene group, an arylene group, and those in which some carbon atoms are replaced with a heteroatom.

As used herein, the term “alkyl group” refers to a linear or branched monovalent saturated aliphatic hydrocarbon group, and examples thereof include a methyl group, an ethyl group, a propyl group, an isobutyl group, a sec-butyl group, a tert-butyl group, a pentyl group, an iso-amyl group, and a hexyl group. As used herein, the term “alkylene group” refers to a linear or branched divalent saturated aliphatic hydrocarbon group, and examples thereof include a methylene group, an ethylene group, a propylene group, a butylene group, and an isobutylene group.

As used herein, the term “halogenated alkyl group” refers to a group in which at least one hydrogen atom of the alkyl group is substituted with a halogen atom, and examples thereof include CF₃.

As used herein, the term “alkoxy group” refers to a monovalent group represented by formula -OA₁₀₁, where A₁₀₁ is an alkyl group. Examples thereof include a methoxy group, an ethoxy group, and an isopropyloxy group.

As used herein, the term “alkylthio group” refers to a monovalent group represented by formula —SA₁₀₁, where A₁₀₁ is an alkyl group.

As used herein, the term “halogenated alkoxy group” refers to an alkoxy group, at least one hydrogen atom of which is substituted with a halogen atom, and examples thereof include —OCF₃.

As used herein, the term “halogenated alkylthio group” refers to an alkylthio group, at least one hydrogen atom of which is substituted with a halogen atom, and examples thereof include —SCF₃.

As used herein, the term “cycloalkyl group” refers to a monovalent saturated hydrocarbon cyclic group, and examples thereof include a monocyclic group such as a cyclopropyl group, a cyclobutyl group, a cyclopentyl group, a cyclohexyl group, and a cycloheptyl group and a condensed polycyclic group such as a norbonyl group and an adamantly group. As used herein, the term “cycloalkylene group” refers a divalent saturated hydrocarbon cyclic group, and examples thereof include a cyclopentylene group, a cyclohexylene group, an adamantylene group, an adamantylmethylene group, a norbornylene group, a norbornylmethylene group, a tricyclodecanylene group, a tetracyclododecanylene group, a tetracyclododecanylmethylene group, and a dicyclohexylmethylene group.

As used herein, the term “cycloalkoxy group” refers to a monovalent group represented by formula —OA₁₀₂, where A₁₀₂ is a cycloalkyl group. Examples thereof include a cyclopropoxy group and a cyclobutoxy group.

As used herein, the term “cycloalkylthio group” refers to a monovalent group represented by formula —SA₁₀₂, where A₁₀₂ is a cycloalkyl group.

As used herein, the term “heterocycloalkyl group” refers to a cycloalkyl group in which some carbon atoms are substituted with a moiety including a heteroatom, such as oxygen, sulfur, or nitrogen, and the heterocycloalkyl group may specifically include an ether bond, an ester bond, a sulfonate ester bond, a carbonate, a lactone ring, a sultone ring, or a carboxylic anhydride moiety. As used herein, the term “heterocycloalkylene group” refers to a cycloalkylene group in which some carbon atoms are substituted with a moiety including a heteroatom such as oxygen, sulfur, or nitrogen.

As used herein, the term “heterocycloalkoxy group” refers to a monovalent group represented by formula —OA₁₀₃, where A₁₀₃ is a heterocycloalkyl group.

As used herein, the term “alkenyl group” refers to a linear or branched monovalent unsaturated aliphatic hydrocarbon group including at least one carbon-carbon double bond. As used herein, the term “alkenylene group” refers to a linear or branched divalent saturated aliphatic hydrocarbon group including at least one carbon-carbon double bond.

As used herein, the term “alkenyloxy group” refers to a monovalent group represented by formula —OA₁₀₄, where A₁₀₄ is an alkenyl group.

As used herein, the term “cycloalkenyl group” refers to a monovalent unsaturated hydrocarbon cyclic group including at least one carbon-carbon double bond. As used herein, the term “cycloalkenylene group” refers to a divalent unsaturated hydrocarbon cyclic group including at least one carbon-carbon double bond.

As used herein, the term “cycloalkenyloxy group” refers to a monovalent group represented by formula —OA₁₀₅, where A₁₀₅ is a cycloalkenyl group.

As used herein, the term “heterocycloalkenyl group” refers to a cycloalkenylene group in which some carbon atoms are substituted with a moiety including a heteroatom, such as oxygen, sulfur, or nitrogen. As used herein, the term “heterocycloalkenylene group” refers to a cycloalkenylene group in which some carbon atoms are substituted with a moiety including a heteroatom, such as oxygen, sulfur, or nitrogen.

As used herein, the term “heterocycloalkenyloxy group” refers to a monovalent group represented by formula —OA₁₀₆, where A₁₀₆ is a heterocycloalkenyl group.

As used herein, the term “alkynyl group” refers to a linear or branched monovalent unsaturated aliphatic hydrocarbon group including at least one carbon-carbon triple bond.

As used herein, the term “alkynyloxy group” refers to a monovalent group represented by formula —OA₁₀₇, where A₁₀₇ is an alkynyl group.

As used herein, the term “aryl group” refers to a monovalent group including a carbocyclic aromatic system, and examples thereof include a phenyl group, a naphthyl group, an anthracenyl group, a phenanthrenyl group, a pyrenyl group, and a chrysenyl group.

As used herein, the term “aryloxy group” refers to a monovalent group represented by formula —OA₁₀₈, where A₁₀₈ is an aryl group.

As used herein, the term “heteroaryl group” refers to a monovalent group including a heterocyclic aromatic system, and examples thereof include a pyridinyl group, a pyrimidinyl group, and a pyrazinyl group. As used herein, the term “heteroarylene group” refers to a divalent group including a heterocyclic aromatic system.

As used herein, the term “heteroaryloxy group” refers to a monovalent group represented by formula —OA₁₀₉, where A₁₀₉ is a heteroaryl group.

As used herein, the term “substituent” includes deuterium, a halogen atom, a hydroxyl group, a cyano group, a nitro group, a carbonyl group, a carboxylate group, an amino group, an ether moiety, an ester moiety, a sulfonate ester moiety, a carbonate moiety, a carbamate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₅-C₂₀ cycloalkyl group, a C₃-C₂₀ cycloalkoxy group, a C₅-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, or a C₁-C₂₀ heteroarylthio group; and

a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₅-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, and a C₁-C₂₀ heteroarylthio group; and any combination thereof, which are unsubstituted or substituted with deuterium, a halogen atom, a hydroxyl group, a cyano group, a nitro group, a carbonyl group, a carboxylate group, an amino group, an ether moiety, an ester moiety, a sulfonate ester moiety, a carbonate moiety, a carbamate moiety, an amide moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₁-C₂₀ alkylthio group, a C₁-C₂₀ halogenated alkoxy group, a C₁-C₂₀ halogenated alkylthio group, a C₃-C₂₀ cycloalkyl group, a C₃-C₂₀ cycloalkoxy group, a C₃-C₂₀ cycloalkylthio group, a C₆-C₂₀ aryl group, a C₆-C₂₀ aryloxy group, a C₆-C₂₀ arylthio group, a C₁-C₂₀ heteroaryl group, a C₁-C₂₀ heteroaryloxy group, a C₁-C₂₀ heteroarylthio group, and any combination thereof.

Hereinafter, embodiments will be described in detail with reference to the accompanying drawings. In the drawings, like reference numerals denote like elements or components having substantially same functions, and duplicate descriptions will be omitted. In the drawings, thicknesses of various layers and regions are enlarged for clarity. In the drawings, for convenience of explanation, thicknesses of some layers and regions are exaggerated for clarity. Meanwhile, it should be understood that embodiments described hereinafter are merely for illustrative purposes, various changes in form from the embodiments may be made.

### [Polymer]

A polymer according to embodiments includes a first repeating unit represented by Formula 1 below.

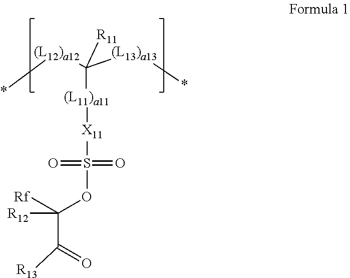

In Formula 1,
L₁₁ to L₁₃ are each independently a single bond; O; S; C(═O); C(═O)O; OC(═O); C(═O)NH; NHC(═O); or a linear, branched, or cyclic C₁-C₃₀ divalent hydrocarbon group optionally including a heteroatom,
a11 to a13 are each independently an integer from 1 to 4,
X₁₁ is a linear, branched, or cyclic C₁-C₃₀ divalent hydrocarbon group optionally including a heteroatom other than F,
Rf is a linear, branched, or cyclic C₁-C₃₀ monovalent hydrocarbon group including at least one F and optionally including a heteroatom other than F,
R₁₁ and R₁₂ are each independently hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
R₁₃ is a substituted or unsubstituted C₆-C₃₀ aryl group or a substituted or unsubstituted C₁-C₃₀ heteroaryl group,
an adjacent two among Rf, R₁₂ optionally may bind to each other to form a ring, and
* is a binding site with an adjacent atom.

The polymer may not include a repeating unit, the structure of which is changed by an acid (e.g., an acid labile group).

In this regard, the repeating unit, the structure of which is changed by an acid refers to a repeating unit including an acid labile group. The acid labile group may refer to a group including a tertiary acyclic alkyl carbon, a group including a tertiary alicyclic carbon, or acetal. More specifically, the acid labile group may refer to an ester group including a tertiary acyclic alkyl carbon, an ester group including a tertiary alicyclic carbon, a carbonate group including a tertiary acyclic alkyl carbon, a carbonate group including a tertiary alicyclic carbon, a carbamate group including a tertiary acyclic alkyl carbon, a carbamate group including a tertiary alicyclic carbon, or acetal, for example, may be a group represented by one of Formulae 6-1 to 6-10 below.

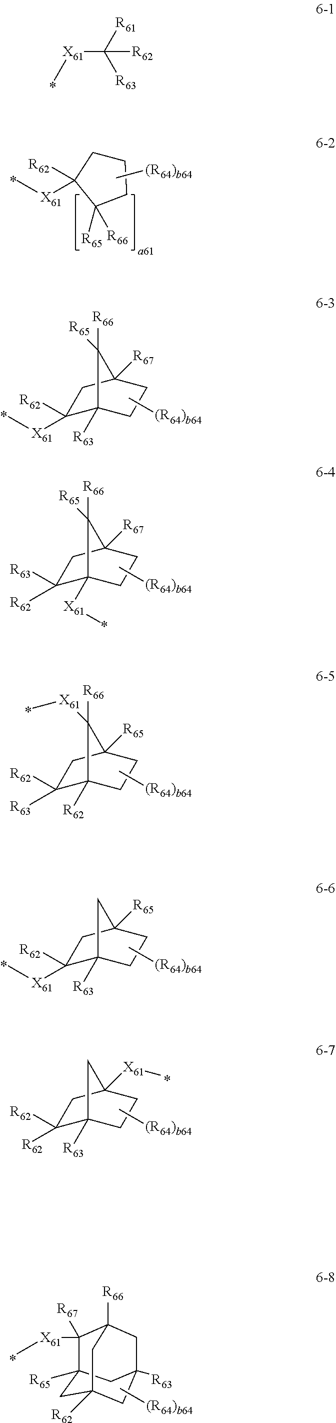

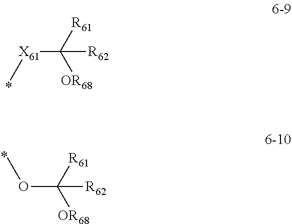

In Formulae 6-1 to 6-10,
X₆₁ is an ester moiety, a carbonate moiety, or a carbamate moiety;
a61 is selected from integers of 0 to 6;
R₆₁ and R₆₈ are each independently a linear, branched, or cyclic C₁-C₂₀ monovalent hydrocarbon group optionally including a heteroatom;
R₆₂ to R₆₇ are each independently hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
among R₆₁ to R₆₈, two adjacent ones optionally bind to each other to form a ring,
b64 is selected from integers of 1 to 10, and
* is a binding site with an adjacent atom.

The acid labile group is detached from the polymer by an acid allowing the polymer to be more easily dissolved in a developer, such as, a TMAH (tetramethylammonium hydroxide) aqueous solution.

For example, in Formula 1, L₁₁ to L₁₃ may be each independently a single bond; O; S; C(═O); C(═O)O; OC(═O); C(═O)NH; NHC(═O); a substituted or unsubstituted C₁-C₃₀ alkylene group; a substituted or unsubstituted C₃-C₃₀ cycloalkylene group; a substituted or unsubstituted C₃-C₃₀ heterocycloalkylene group; a substituted or unsubstituted C₂-C₃₀ alkenylene group; a substituted or unsubstituted C₃-C₃₀ cycloalkenylene group; a substituted or unsubstituted C₃-C₃₀ heterocycloalkenylene group; a substituted or unsubstituted C₆-C₃₀ arylene group; or a substituted or unsubstituted C₁-C₃₀ heteroarylene group.

As another example, in Formula 1, Li to L₁₃ may be each independently selected from a single bond; O; C(═O); C(═O)O; OC(═O); C(═O)NH; NHC(═O); and a C₁-C₂₀ alkylene group, a C₃-C₂₀ cycloalkylene group, a C₅-C₂₀ heterocycloalkylene group, a C₂-C₂₀ alkenylene group, a C₃-C₂₀ cycloalkenylene group, a C₅-C₂₀ heterocycloalkenylene group, a C₆-C₂₀ arylene group, and a C₁-C₂₀ heteroarylene group, which are unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₅-C₂₀ cycloalkyl group, a C₃-C₂₀ cycloalkoxy group, a C₆-C₂₀ aryl group, or any combination thereof.

As another example, in Formula 1, L₁₁ to Lis may be each independently selected from a single bond; O; C(═O); C(═O)O; OC(═O); C(═O)NH; NHC(═O); and a C₁-C₂₀ alkylene group, a C₃-C₂₀ cycloalkylene group, a C₅-C₂₀ heterocycloalkylene group, a phenylene group, and a naphthylene group, which are unsubstituted or substituted with deuterium, a halogen atom, a C₁-C₂₀ alkyl group, a C₁-C₂₀ halogenated alkyl group, a C₁-C₂₀ alkoxy group, a phenyl group, a naphthyl group, or any combination thereof.

In Formula 1, a11 to a13 refer to numbers of repetition of L₁₁ to L₁₃, respectively.

For example, in Formula 1, a11 to a13 are each independently an integer from 1 to 3.

As another example, in Formula 1, a11 to a13 are each independently 1.

For example, in Formula 1, X₁₁ may be a linear, branched, or cyclic C₁-C₃₀ divalent hydrocarbon group optionally including O, S, N, or any combination thereof.

As an example, in Formula 1, X₁₁ may be selected from a C₁-C₂₀ alkylene group, a C₃-C₂₀ cycloalkylene group, a C₂-C₂₀ alkenylene group, a C₃-C₂₀ cycloalkenylene group, and a C₆-C₂₀ arylene group, which are unsubstituted or substituted with deuterium, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ alkoxy group, a C₅-C₂₀ cycloalkyl group, a C₅-C₂₀ cycloalkoxy group, a C₆-C₂₀ aryl group, or any combination thereof.

As another example, in Formula 1, X₁₁ may be selected from a C₁-C₂₀ alkylene group, a C₃-C₂₀ cycloalkylene group, a phenylene group, and a naphthylene group, which are unsubstituted or substituted with deuterium, a C₁-C₂₀ alkyl group, a C₁-C₂₀ alkoxy group, a phenyl group, a naphthyl group, or any combination thereof.

For example, in Formula 1, Rf may be a linear, branched, or cyclic C₁-C₃₀ monovalent hydrocarbon group including at least one F and optionally including O.

As an example, in Formula 1, Rf may be selected from a C₁-C₂₀ alkyl group substituted with at least one F, a C₁-C₂₀ alkoxy group substituted with at least one F, a C₃-C₂₀ cycloalkyl group substituted with at least one F, and a C₃-C₂₀ cycloalkoxy group substituted with at least one F.

As another example, in Formula 1, Rf may be a C₁-C₂₀ alkyl group substituted with at least one F

As another example, in Formula 1, Rf may be CH₂F, CHF₂, CF₃, CHFCH₃, CHFCH₂F, CHFCHF₂, CHFCF₃, CF₂CH₃, CF₂CH₂F, CF₂CHF₂, or CF₂CF₃.

For example, in Formula 1, R₁₁ and R₁₂ may be each independently selected from hydrogen; deuterium; a halogen atom; a cyano group; a hydroxyl group; an amino group; a carboxylate group; a thiol group; and a C₁-C₂₀ alkyl group, a C₅-C₂₀ cycloalkyl group, and a C₆-C₂₀ aryl group, which are unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

As another example, in Formula 1, R₁₁ may be hydrogen, deuterium, a halogen atom, CH₃, CH₂F, CHF₂, CF₃, CH₂CH₃, CHFCH₃, CHFCH₂F, CHFCHF₂, CHFCF₃, CF₂CH₃, CF₂CH₂F, CF₂CHF₂, or CF₂CF₃.

As another example, in Formula 1, R₁₂ may be hydrogen; deuterium; a halogen atom; a cyano group; a hydroxyl group; an amino group; a carboxylate group; a thiol group; a C₁-C₂₀ alkyl group; a C₁-C₂₀ halogenated alkyl group; C₅-C₂₀ cycloalkyl group; or a C₆-C₂₀ aryl group.

For example, in Formula 1, R₁₃ may be selected from a C₆-C₃₀ aryl group and a C₁-C₃₀ heteroaryl group, which are unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₅-C₂₀ cycloalkoxy group, a C₆-C₂₀ aryl group, or any combination thereof.

As another example, in Formula 1, R₁₃ may be selected from a phenyl group, a biphenyl group, a terphenyl group, a naphthyl group, a phenanthrenyl group, an anthracenyl group, a triphenylenyl group, a pyrenyl group, a chrysenyl group, a pyrrolyl group, a thiophenyl group, a furanyl group, an imidazolyl group, a pyrazolyl group, a thiazolyl group, an oxazolyl group, a pyridinyl group, a pyrazinyl group, a pyrimidinyl group, a pyridazinyl group, an indolyl group, a quinolinyl group, an isoquinolinyl group, a benzoquinolinyl group, a quinoxalinyl group, a quinazolinyl group, and a cinnolinyl group, which are unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

As another example, in Formula 1, R₁₃ may be selected from a phenyl group, a biphenyl group, a terphenyl group, and a naphthyl group, which are unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₅-C₂₀ cycloalkoxy group, a C₆-C₂₀ aryl group, or any combination thereof.

For example, in Formula 1, Rf and R₁₂, R₁₂ and R₁₃, or Rf and R₁₃ may optionally bine to each other to form a ring.

According to an embodiment, the first repeating unit may be represented by Formula 1-1 below.

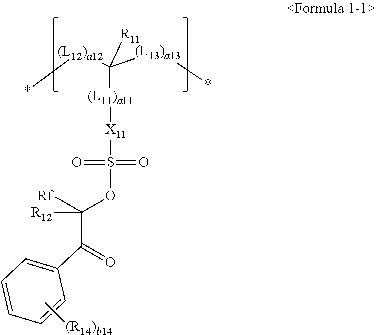

In Formula 1-1,
L₁₁ to L₁₃, a11 to a13, X₁₁, Rf and R₁₁ to R₁₃ are as defined above in Formula 1,
R₁₄ is hydrogen, deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, or a C₆-C₂₀ aryl group,
b14 is an integer from 1 to 5, and
* is a binding site with an adjacent atom.

Specifically, the first repeating unit may be selected from compounds of Group I below.

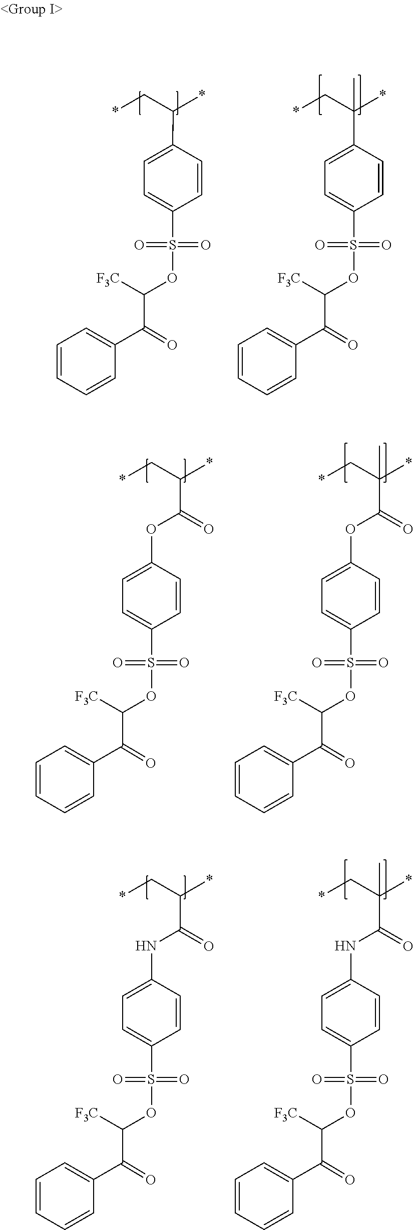

According to an embodiment, the polymer may further include a second repeating unit represented by Formula 2 below.

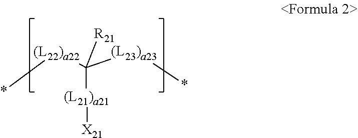

In Formula 2,
L₂₁ to L₂₃ are each independently a single bond; O; S; C(═O); C(═O)O; OC(═O); C(═O)NH; NHC(═O); or a linear, branched, or cyclic C₁-C₃₀ divalent hydrocarbon group optionally including a heteroatom,
a21 to a23 are each independently an integer from 1 to 4,
R₂₁ is hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety; a sultone moiety; a carboxylic anhydride moiety; or a linear, branched, or cyclic C₁-C₃₀ monovalent hydrocarbon group optionally including a heteroatom,
X₂₁ is a non-acid labile group, and
* is a binding site with an adjacent atom.

Specifically, in Formula 2, L₂₁ to L₂₃ are each independently as defined in descriptions of L₁₁.

Specifically, in Formula 2, a21 to a23 are each independently as defined in descriptions of a11.

Specifically, in Formula 2, R₂₁ is as defined in descriptions of R₁₁.

Specifically, in Formula 2, X₂₁ may be hydrogen; a halogen atom; a cyano group; a hydroxyl group; a carboxylate group; a thiol group; an amino group; or a linear, branched, or cyclic C₁-C₃₀ monovalent hydrocarbon group optionally including at least one polar moiety selected from a halogen atom, a cyano group, a hydroxyl group, a thiol group, a carboxylate group, O, C═O, C(═O)O, OC(═O), S(═O)O, OS(═O), a lactone moiety, a sultone moiety, and a carboxylic anhydride moiety.

According to an embodiment, in Formula 2, X₂₁ may be hydrogen, a hydroxyl group, or a group represented by one of Formulae 5-1 to 5-12 below.

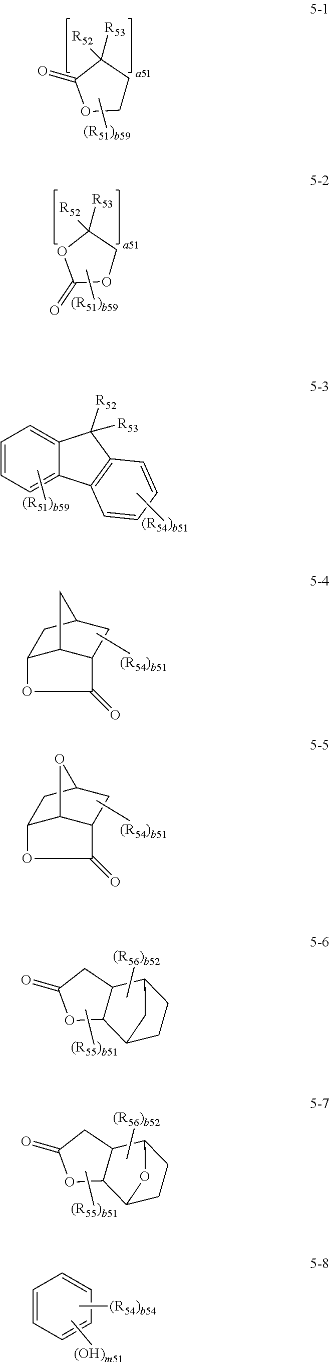

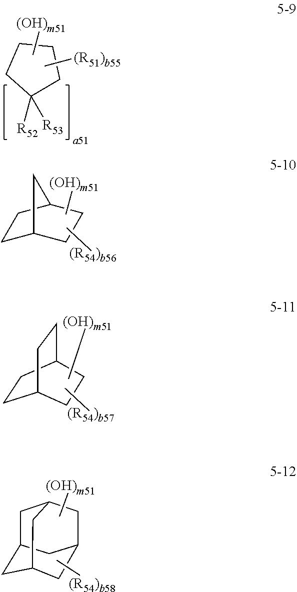

In Formulae 5-1 to 5-12,
a51 is 1 or 2;
R₅₁ to R₅₆ are each independently a binding site with an adjacent atom; hydrogen; a hydroxyl group; a carboxylate group; an ester moiety; a carbonate moiety; a carbamate moiety; a lactone moiety; a carboxylic anhydride moiety; or a linear, branched, or cyclic C₁-C₂₀ monovalent hydrocarbon group,
one of R₅₁(s), R₅₂(s), and R₅₃(s), one of R₅₄(s), and one of R₅₅(s) and R₅₆(s) are binding sites with an adjacent atom,
b51 is selected from integers of 1 to 4,
b52 is selected from integers of 1 to 10,
b53 is selected from integers of 1 to 8,
b54 is selected from integers of 1 to 5,
b55 is selected from integers of 1 to 7,
b56 is selected from integers of 1 to 11,
b57 is selected from integers of 1 to 13,
b58 is selected from integers of 1 to 15,
b59 is selected from integers of 1 and 2, and
m51 is selected from integers of 1 to 4.

Particularly, in Formula 2, X₂₁ may be selected from a hydroxyl group and groups represented by Formulae 5-1, 5-6, and 5-10 above.

According to an embodiment, the second repeating unit may be represented by one or more formulae selected from Formula 2-1 below.

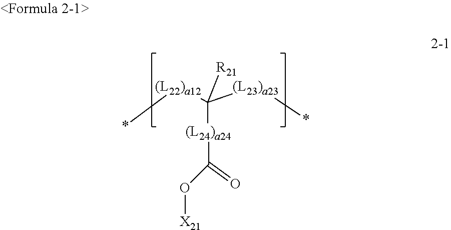

In Formula 2-1,
L₂₂ to L₂₃, a22 to a23, R₂₁ and X₂₁ are as described in Formula 2,
L₂₄ is a single bond; O; S; C(═O); C(═O)O; OC(═O); C(═O)NH; NHC(═O); or a linear, branched, or cyclic C₁-C₃₀ divalent hydrocarbon group optionally including a heteroatom,
a24 is an integer from 1 to 3, and
* is a binding site with an adjacent atom.

Particularly, in Formula 2-1, X₂₁ may be selected from a hydroxyl group and groups represented by Formulae 5-1, 5-6, and 5-10 above.

In an embodiment, the second repeating unit may be represented by one or more formulae selected from compounds of Group II below.

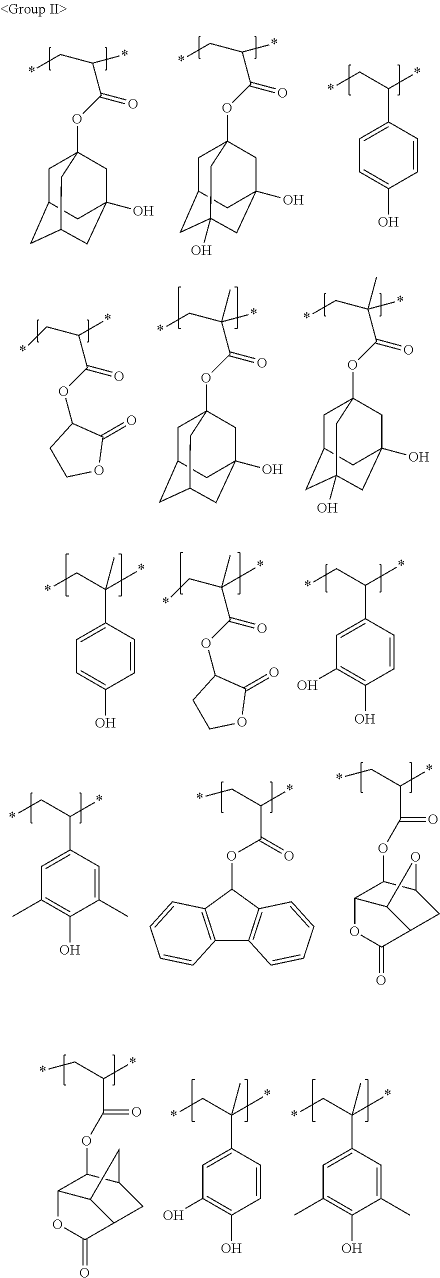

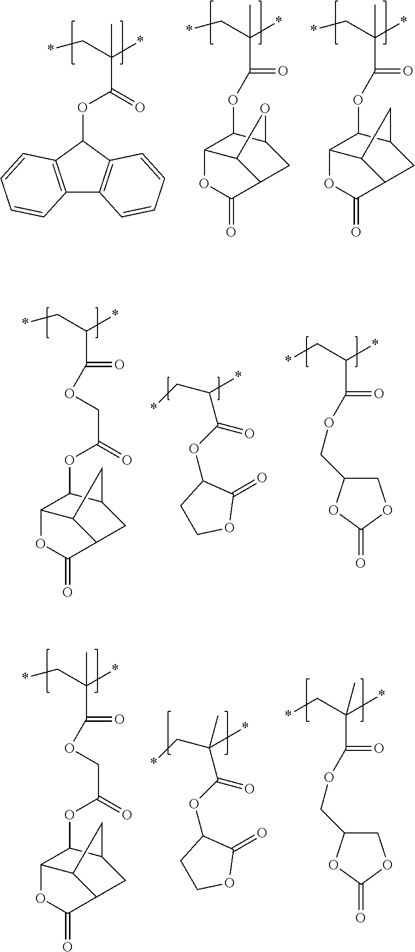

In an embodiment, the polymer may consist of the first repeating unit. The polymer may include the first repeating unit in an amount of 1 to 100 mol %, more specifically, 5 to 100 mol %, particularly, 10 to 100 mol %.

In another embodiment, the polymer may consist of the first repeating unit and the second repeating unit. Specifically, the polymer may include the first repeating unit in an amount of 1 to 90 mol % and the second repeating unit in an amount of 10 to 99 mol %. More specifically, the polymer may include the first repeating unit in an amount of 10 to 80 mol % and the second repeating unit in an amount of 20 to 90 mol %. Particularly, the polymer may include the first repeating unit and the second repeating unit in a molar ratio of 5:1 to 1:5.

The polymer may have a weight average molecular weight Mw of 1,000 to 500,000, specifically, 3,000 to 200,000, measured by gel permeation chromatography using a tetrahydrofuran solvent and polystyrene as standard materials.

The polymer may have a polydipersity index (PDI: Mw/Mn) of 1.0 to 3.0. If the above-described range is satisfied, dispersity and/or compatibility of the polymer may be easily controlled, a possibility of foreign substances remaining on the pattern may be reduced, or deterioration of a pattern profile may be minimized. Accordingly, the resist composition may become more suitable for forming fine patterns.

Because physical properties of the polymer itself may change by high-energy rays, the polymer may be used in non-chemically amplified resist compositions.

The polymer has relatively high resistance to oxygen and/or moisture and have physical properties changed only by high-energy rays, and thus a resist composition having improved physical properties such as storage stability may be provided.

Because a change in physical properties of the polymer is induced by a structural change of a side chain, compared to a system configured to induce physical properties by decomposition a main chain of a polymer, a resist composition enabling patterning with increased resolution, improved line edge roughness (LER), and/or enhanced line width roughness (LWR) may be provided even at a low dose of high-energy rays.

Particularly, unlike chemically amplified photoresists, which may cause problems such as low uniformity of a pattern or high surface roughness due to the produced acid diffused to an unexposed region, the solubility of the polymer is not changed by the acid, and thus the problems of low uniformity of the pattern and/or generation of defects caused by diffusion of the acid may be reduced.

The polymer may be manufactured by any appropriate methods, for example, by a method of dissolving monomer(s) containing unsaturated bonds in an organic solvent and performing thermal polymerization in the presence of a radical initiator.

The structure (composition) of the polymer may be identified by FT-IR analysis, NMR analysis, X-ray fluorescence (XRF) analysis, mass spectrometry, UV analysis, single crystal X-ray diffraction, powder X-ray diffraction (PXRD), liquid chromatography (LC), size-exclusion chromatography (SEC), thermal analysis, or the like. Detailed descriptions for identification methods therefor are as shown in the examples below.

### [Monomer]

A monomer according to embodiments is represented by Formula 10 below.

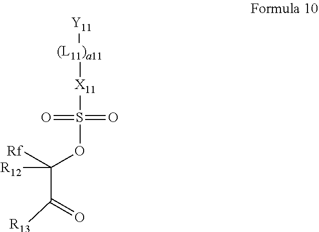

In Formula 10,
Y₁₁ is a polymerizable group;
L₁₁ is a single bond; O; S; C(═O); C(═O)O; OC(═O); C(═O)NH; NHC(═O); or a linear, branched, or cyclic C₁-C₃₀ divalent hydrocarbon group optionally including a heteroatom,
a11 is an integer from 1 to 4,
X₁₁ is a linear, branched, or cyclic C₁-C₃₀ divalent hydrocarbon group optionally including a heteroatom other than F,
Rf is a linear, branched, or cyclic C₁-C₃₀ monovalent hydrocarbon group including at least one F and optionally including a heteroatom other than F,
R₁₂ is hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety; a sultone moiety; a carboxylic anhydride moiety; or a linear, branched, or cyclic C₁-C₃₀ monovalent hydrocarbon group optionally including a heteroatom,
R₁₃ is a substituted or unsubstituted C₆-C₃₀ aryl group or a substituted or unsubstituted C₁-C₃₀ heteroaryl group, and
an adjacent two among Rf, R₁₂, and R₁₃ optionally may bind to each other to form a ring, and

In Formula 10, for detailed descriptions of L₁₁, a11, X₁₁, Rf, R₁₂, and R₁₃, refer to descriptions of Formula 1.

For example, in Formula 10, Y₁₁ may include a vinyl group, an acrylate group, a methacrylate group, an oxirane group, an epoxy group, an oxetane group, a thiol group, or any combination thereof as a partial structure.

Specifically, in Formula 10, Y₁₁ may include a vinyl group, an acrylate group, a methacrylate group, or any combination thereof as a partial structure.

In an embodiment, the monomer represented by Formula 10 may be selected from compounds of Group III below.

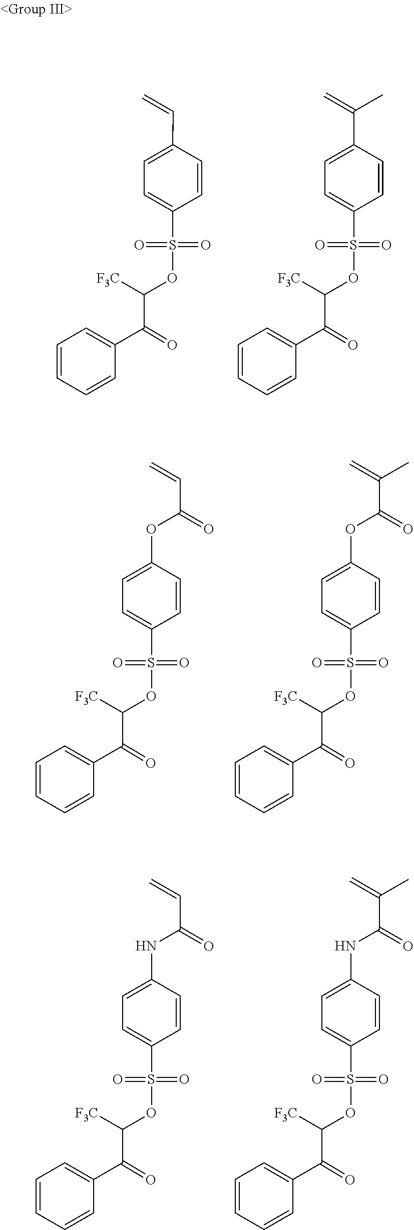 [SMILES: C=Cc1ccc(S(=O)(=O)OC(C(=O)c2ccccc2)C(F)(F)F)cc1 ; C=C(C)c1ccc(S(=O)(=O)OC(C(=O)c2ccccc2)C(F)(F)F)cc1 ; C=CC(=O)Oc1ccc(S(=O)(=O)OC(C(=O)c2ccccc2)C(F)(F)F)cc1 ; C=C(C)C(=O)Oc1ccc(S(=O)(=O)OC(C(=O)c2ccccc2)C(F)(F)F)cc1 ; C=CC(=O)Nc1ccc(S(=O)(=O)OC(C(=O)c2ccccc2)C(F)(F)F)cc1 ; C=C(C)C(=O)Nc1ccc(S(=O)(=O)OC(C(=O)c2ccccc2)C(F)(F)F)cc1]

### [Resist Composition]

According to another aspect, a resist composition includes the above-described polymer and an organic solvent. The resist composition may have enhanced the compounds of Group characteristics such as improved developability and/or increased resolution.

Exposure to high-energy rays changes solubility of the resist composition in a developer. The resist composition may be a positive resist composition to form a positive resist pattern by dissolving and removing an exposed portion, of a negative resist composition to form negative resist pattern. Also, the resist composition according to an embodiment may be one for an alkaline developing process using an alkaline developer for development while forming a resist pattern or one for a solvent developing process using an organic solvent-containing developer (hereinafter, referred to as organic developer) for development.

Because the resist composition is a non-chemically amplified type, the composition may not substantially include a photoacid generator.

The resist composition may not substantially include a compound having a molecular weight of 1,000 or more in addition to the polymer because physical properties of the polymer are changed by light exposure.

The polymer may be used in an amount of 0.1 to 80 parts by weight based on 100 parts by weight of the resist composition. Specifically, the polymer may be used in an amount of 0.5 to 5 parts by weight based on 100 parts by weight of the resist composition. If the above-described range is satisfied, loss of performance, e.g., a decrease in sensitivity and/or formation of particles of foreign matter caused by insufficient solubility may be reduced.

Because the polymer is as described above, hereinafter, the organic solvent and optional components included if required will be described. In addition, the polymer used may be used alone in the resist composition or at least two thereof may be used in combination.

According to another aspect, a resist composition includes the above-described monomer and an organic solvent. The resist composition may have enhanced characteristics such as improved developability and/or increased resolution.

The resist composition including the monomer is the same as the resist composition including the above-described polymer except that the resist composition includes the monomer instead of the polymer. For descriptions of the resist composition including the monomer, refer to descriptions of the resist composition including the polymer given above.

### <Organic Solvent>

The organic solvent included in the resist composition is not particularly limited, as long as the polymer and any component contained therein, if required, may be dissolved or dispersed therein. The organic solvent may be used alone or any combination of two or more different organic solvents may also be used. Also, a mixed solvent of water and an organic solvent may be used.

Examples of the organic solvent may include alcohol-based solvents, ether-based solvents, ketone-based solvents, amide-based solvents, ester-based solvents, sulfoxide-based solvents, and hydrocarbon-based solvents.

More specifically, examples of the alcohol-based solvents may include: a monoalcohol-based solvent such as methanol, ethanol, n-propanol, isopropanol, 1-methoxy-2-propanol, 1-ethoxy-2-propanol, n-butanol, isobutanol, sec-butanol, tert-butanol, n-pentanol, isopentanol, 2-methylbutanol, sec-pentanol, tert-pentanol, 3-methoxybutanol, 3-methyl-3-methoxy butanol, n-hexanol, 2-methylpentanol, sec-hexanol, 2-ethylbutanol, 4-methyl-2-pentanol (MIBC), sec-heptanol, 3-heptanol, n-octanol, 2-ethylhexanol, sec-octanol, n-nonylalcohol, 2,6-dimethyl-4-heptanol, n-decanol, sec-undecyl alcohol, trimethylnonylalcohol, sec-tetradecyl alcohol, sec-heptadecyl alcohol, furfuryl alcohol, phenol, cyclohexanol, methylcyclohexanol, 3,3,5-trimethylcyclohexanol, benzyl alcohol, and diacetone alcohol; a polyalcohol-based solvent such as ethyleneglycol, 1,2-propylene glycol, 1,3-butylene glycol, 2,4-pentanediol, 2-methyl-2,4-pentanediol, 2,5-hexanediol, 2,4-heptanediol, 2-ethyl-1,3-hexanediol, diethyleneglycol, dipropyleneglycol, triethylene glycol, and tripropylene glycol; and a polyalcohol-containing ether-based solvent such as ethyleneglycol monomethylether, ethyleneglycol monoethylether, ethyleneglycol monopropylether, ethyleneglycol monobutylether, ethyleneglycol monohexylether, ethyleneglycol monophenylether, ethyleneglycol mono-2-ethylbutylether, diethyleneglycol monomethylether, diethyleneglycol monoethylether, diethyleneglycol monopropylether, diethyleneglycol monobutylether, diethyleneglycol monohexyl ether, diethylene glycol dimethylether, propylene glycol monomethylether, propylene glycol dimethylether, propylene glycol monoethylether, propylene glycol monopropylether, propylene glycol monobutylether, dipropyleneglycol monomethylether, dipropyleneglycol monoethylether, and dipropyleneglycol monopropylether.

Examples of the ether-based solvents may include: a dialkylether-based solvent such as diethylether, dipropylether, and dibutylether; a cyclic ether-based solvent such as tetrahydrofuran and tetrahydropyran; and an aromatic ring-containing ether-based solvent such as diphenylether and anisole.

Examples of the ketone-based solvents may include: a chain-shaped ketone-based solvent such as acetone, methylethylketone, methyl-n-propylketone, methyl-n-butylketone, methyl-n-pentylketone, diethylketone, methylisobutylketone, 2-heptanone, ethyl-n-butylketone, methyl-n-hexylketone, diisobutylketone, and trimethylnonanone; a cyclic ketone-based solvent such as cyclopentanone, cyclo hexanone, cycloheptanone, cyclooctanone, and methylcyclohexanone; and 2,4-pentanedione, acetonylacetone, and acetphenone.

Examples of the amide-based solvents may include: a cyclic amide-based solvent such as N,N′-dimethylimidazolidinone and N-methyl-2-pyrrolidone; and a chain-shaped amide-based solvent such as N-methylformamide, N,N-dimethylformamide, N,N-diethylformamide, acetamide, N-methylacetamide, N,N-dimethylacetamide, and N-methylpropyoneamide.

Examples of the ester-based solvents include: an acetate ester-based solvent such as methyl acetate, ethyl acetate, n-propyl acetate, isopropyl acetate, n-butyl acetate, isobutyl acetate, sec-butyl acetate, T-butyl acetate, n-pentyl acetate, isopentyl acetate, sec-pentyl acetate, 3-methoxybutyl acetate, methylpentyl acetate, 2-ethylbutyl acetate, 2-ethylhexyl acetate, benzyl acetate, cyclohexyl acetate, methylcyclohexyl acetate, and n-nonyl acetate; a polyalcohol-containing ethercarboxylate-based solvent such as ethyleneglycol monomethylether acetate, ethyleneglycol monoethylether acetate, diethyleneglycol monomethylether acetate, diethyleneglycol monoethylether acetate, diethyleneglycol mono-n-butyl ether acetate, propylene glycol monomethylether acetate (PGMEA), propylene glycol monoethylether acetate, propylene glycol monopropylether acetate, propylene glycol monobutylether acetate, dipropylene glycol monomethylether acetate, and dipropylene glycol monoethylether acetate; a lactone-based solvent such as γ-butyrolactone and δ-valerolactone; a carbonate-based solvent such as dimethyl carbonate, diethyl carbonate, ethylene carbonate, and propylene carbonate; a lactate ester-based solvent such as methyl lactate, ethyl lactate, n-butyl lactate, and n-amyl lactate; and glycoldiacetate, methoxytriglycol acetate, ethyl propionate, n-butyl propionate, isoamyl propionate, diethyloxalate, di-n-butyloxalate, methyl acetoacetate, ethyl acetoacetate, diethyl malonate, dimethyl phthalate, and diethyl phthalate.

Examples of the sulfoxide-based solvents may include dimethyl sulfoxide and diethyl sulfoxide.

Examples of the hydrocarbon-based solvents include: an aliphatic hydrocarbon-based solvent such as n-pentane, isopentane, n-hexane, isohexane, n-heptane, isoheptane, 2,2,4-trimethyl pentane, n-octane, isooctane, cyclohexane, and methylcyclohexane; and an aromatic hydrocarbon-based solvent such as benzene, toluene, xylene, mesitylene, ethylbenzene, trimethylbenzene, methylethylbenzene, n-propylbenzene, isopropylbenzene, diethylbenzene, isobutylbenzene, triethylbenzene, diisopropylbenzene, and n-amylnaphthalene.

Specifically, the organic solvent may be selected from the alcohol-based solvent, the amide-based solvent, the ester-based solvent, the sulfoxide-based solvent, and any combination thereof. More specifically, the solvent may be selected from propylene glycol monomethylether, propylene glycol monoethylether, propylene glycol monomethylether acetate, N-methyl-2-pyrrolidone, N,N-dimethylacetamide, ethyl lactate, dimethylsulfoxide, and any combination thereof.

The organic solvent may be included in an amount of 200 parts by weight to 5,000 parts by weight, specifically, 400 parts by weight to 3,000 parts by weight based on 100 parts by weight of the polymer.

### <Optional Components>

The resist composition may further include a surfactant, a crosslinking agent, a leveling agent, a colorant, or any combination thereof, if necessary.

The resist composition may further include a surfactant to improve coatablity, developability, and the like. Examples of the surfactant may include a non-ionic surfactant such as polyoxyethylene lauryl ether, polyoxyethylene stearyl ether, polyoxyethyleneoleyl ether, polyoxyethylene n-octylphenyl ether, polyoxyethylene n-nonylphenyl ether, polyethyleneglycol dilaurate, and polyethyleneglycol distearate. Any commercially available product or a synthetic product may be used as the surfactant. Examples of the commercially available product may include KP341 (manufactured by Shin-Etsu Chemical Co., Ltd.), Polyflow No. 75 and Polyflow No. 95 (manufactured by Kyoeisha Chemical Co., Ltd.), Eftop EF301, Eftop EF303, and Eftop EF352 (manufactured by Mitsubishi Materials Electronic Chemicals Co., Ltd.), MEGAFACE F171, MEGAFACE F173, R40, R41, and R43 (manufactured by DIC Corporation), Fluorad® FC430, Fluorad FC431 (manufactured by 3M Co., Ltd.), AsahiGuard AG710 (manufactured by AGC Co., Ltd.), and Surflon® S-382, Surflon SC-101, Surflon SC-102, Surflon SC-103, Surflon SC-104, Surflon SC-105, and Surflon SC-106 (manufactured by AGC Seimi Chemical Co., Ltd).

The surfactant may be included in an amount of 0 parts by weight to 20 parts by weight based on 100 parts by weight of the polymer. The surfactant may be used alone or any mixture of two or more different surfactants may also be used.

A method of preparing the resist composition is not particularly limited, and any method of mixing the polymer and optional components added as occasion demands in an organic solvent may also be used. Temperature or time in the mixing is not particularly limited. If necessary, filtration may be performed after the mixing.

### [Method of Forming Pattern]

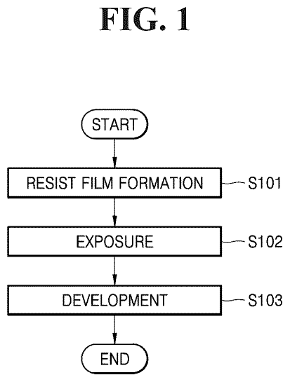

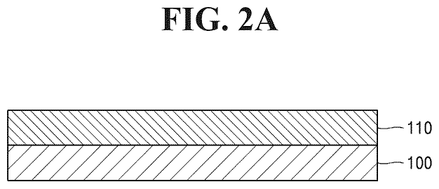

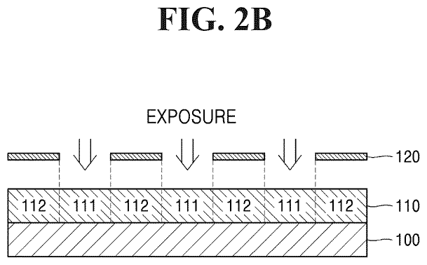

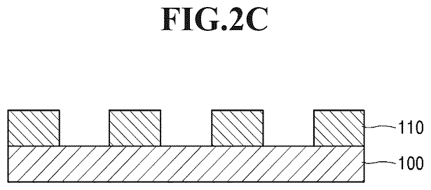

Hereinafter, a method of forming a pattern according to embodiments will be described in more detail with reference to FIGS. 1 and FIGS. 2A to 2C. FIG. 1 is a flowchart illustrating a method of forming a pattern according to embodiments. FIGS. 2A to 2C are side cross-sectional views illustrating a method of forming a pattern according to embodiments. Hereinafter, a method of forming a pattern using a positive resist composition will be described by way of example, but the embodiment is not limited thereto.

Referring to FIG. 1, a method of forming a pattern includes forming a resist film by applying a resist composition to a substrate (S101), exposing at least one portion of the resist film to high-energy rays to provide an exposed resist film (S102), and developing the exposed resist film by using a developer (S103). These operations may be omitted or may be performed in a different order, if necessary.

First, a substrate 100 is prepared. The substrate 100 may be a semiconductor substrate such as a silicon substrate and a germanium substrate, or may be formed of glass, quartz, ceramic, copper, or the like. In some embodiments, the substrate 100 may include Group 3-5 compounds, such as GaP, GaAs, and GaSb.

A resist film 110 may be formed on the substrate 100 by applying the resist composition thereto to a desired thickness using a coating method. If necessary, the resist film 110 may be heated (pre-baked (PB or post-annealing baked (PAB)) to remove the organic solvent remaining in the resist film 110.

As the coating method, spin coating, dipping, roller coating, or other common coating methods may be used. Among them, spin coating may particularly be used. The resist film 110 having a desired thickness may be formed by adjusting viscosity, concentration, and/or spin speed of the resist composition. Specifically, the resist film 110 may have a thickness of 10 nm to 300 nm. More specifically, the resist film 110 may have a thickness of 30 nm to 200 nm.

A lower limit of a pre-baking temperature may be 60° C. or higher, specifically, 80° C. or higher. In addition, an upper limit of the pre-baking temperature may be 150° C. or less, specifically, 140° C. or lower. A lower limit of a pre-baking time may be 5 seconds or more, specifically, 10 seconds or more. An upper limit of the pre-baking time may be 600 seconds or less, specifically, 300 seconds or less.

Before applying the resist composition to the substrate 100, a film to be etched (not shown) may be formed on the substrate 100. The film to be etched may refer to a film onto which an image is transferred from a resist pattern to be converted into a pattern. In an embodiment, the film to be etched may be formed to include, for example, an insulating material such as a silicon oxide, a silicon nitride, and a silicon oxynitride. In some embodiments, the film to be etched may be formed to include a conductive material such as a metal, a metal nitride, a metal silicide, and a metal silicide nitride film. In some embodiments, the film to be etched may be formed to include a semiconductor material such as polysilicon.

In an embodiment, an anti-reflection film may further be formed on the substrate 100 to maximize efficiency of the resist. The anti-reflection film may be an organic or inorganic anti-reflection film.

In an embodiment, a protective film may further be formed on the resist film 110 to reduce effects of alkaline impurities included during a process. In addition, in the case of performing immersion lithography, a protective film for immersion lithography may be formed on the resist film 110 to avoid direct contact between an immersion medium and the resist film 110.

Subsequently, at least one portion of the resist film 110 may be exposed to high-energy rays. For example, high-energy rays having passed through a mask 120 may reach at least one portion of the resist film 110. Therefore, the resist film 110 may have exposed regions 111 and unexposed regions 112.

Although not limited to a particular theory, as a side chain is decomposed in the exposed portion 111 by exposure to light, sulfonic acid that is acidic is produced at the side chain of the polymer, and thus solubility of the polymer in a developer, particularly, in an alkaline developer, may increase. Specifically, a reaction as shown in the schematic diagram below may occur.

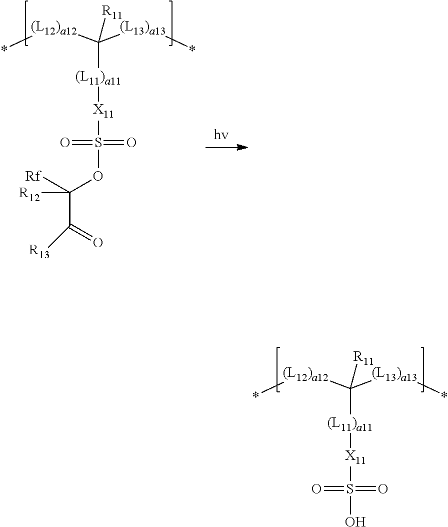

The exposure to light is performed by emitting high-energy rays through a mask having a particular pattern using a liquid such as water as a medium, if necessary. Examples of the high-energy rays may include electromagnetic waves such as ultraviolet rays, deep ultraviolet rays, extreme ultraviolet (EUV) rays (wavelength of 13.5 nm), X-rays, and γ-rays; and charged particle beams such as electron beams and a particle beams. Irradiation of these high-energy rays may be collectively referred to as “exposure”.

Various light sources may be used for the exposure. For example, a light source emitting laser beams in the UV range, such as a KrF excimer laser (wavelength of 248 nm), an ArF excimer laser (wavelength of 193 nm), and an F2 excimer laser (wavelength of 157 nm), a light source emitting harmonic laser beams in the far ultraviolet or vacuum ultraviolet range by converting wavelengths of laser beams received from a solid laser light source (YAG or semiconductor laser), and a light source emitting electron beams or EUVs may be used. During exposure, a mask corresponding to a desired pattern is commonly used. However, in the case of using electron beams as a light source for exposure, exposure may be directly performed without using a mask.

An integral dose of the high-energy rays may be 2000 mJ/cm² or less, specifically, 500 mJ/cm² or less, in the case of using extreme ultraviolet rays as the high-energy rays. In addition, in the case of using electron beams as the high-energy rays, the integral dose may be 5000 μC/cm² or less, specifically, 1000 μC/cm² or less.

Also, post exposure baking (PEB) may be performed after exposure. A lower limit of a PEB temperature may be 50° C. or higher, specifically, 80° C. or higher. An upper limit of the PEB temperature may be 180° C. or lower, specifically, 130° C. or lower. A lower limit of a PEB time may be 5 seconds or more, specifically, 10 seconds or more. An upper limit of the PEB time may be 600 seconds of less, specifically, 300 seconds or less.

The exposed resist film 110 may be developed using a developer. The exposed regions 111 may be washed away by the developer, the unexposed regions 112 may remain without being washed by the developer. Or, on the contrary, the unexposed portion 112 may be washed away by the developer, or the exposed portion 111 may remain without being washed away by the developer.

As the developer, an alkaline developer, a develop including an organic solvent (hereinafter, referred to as “organic developer”), and the like may be used. A developing method may be dipping, puddling, spraying, dynamic approach, or the like. A developing temperature may be, for example, from 5° C. to 60° C., and a developing time may be, for example, from 5 seconds to 300 seconds.

Examples of the alkaline developer may include an alkaline aqueous solution including at least one alkaline compound dissolved therein such as sodium hydroxide, potassium hydroxide, sodium carbonate, sodium silicate, sodium metasilicate, aqueous ammonia, ethylamine, n-propylamine, diethylamine, di-n-propyl amine, triethylamine, methyldiethylamine, ethyldimethylamine, triethanolamine, tetramethyl ammonium hydroxide (TMAH), pyrrole, piperidine, choline, 1,8-diazabicyclo [5.4.0]-7-undecene (DBU), and 1,5-diazabicyclo[4.3.0]-5-nonene (DBN). The alkaline developer may further include a surfactant.

A lower limit of an amount of the alkaline compound contained in the alkaline developer may be 0.1 mass % or more, specifically, 0.5 mass % or less, more specifically, 1 mass % or more. In addition, an upper limit of the amount of the alkaline compound contained in the alkaline developer may be 20 mass % or less, specifically, 10 mass % or less, more specifically, 5 mass % or less.

After development, the resist pattern may be washed with ultrapure water, and then, water remaining on the substrate and the pattern may be removed.

For example, the organic solvent contained in the organic developer may be the same as those described above in the <Organic Solvent> section of the [Resist Composition].

In the organic developer, a lower limit of the amount of the organic solvent may be 80 wt % or more, specifically, 90 wt % or more, more specifically, 95 wt % or more, particularly, 99 wt % or more.

The organic developer may include a surfactant. In addition, the organic developer may include a trace amount of moisture. In addition, development may be stopped during developing by replacing the organic developer with a different type of solvent.

The resist pattern may further be washed after the developing. Ultrapure water, a ringing solution, and the like may be used as a washing solution. The rinsing solution is not particularly limited as long as the resist pattern is not dissolved therein, and any solution including a common organic solvent may be used. For example, the rinsing solution may be an alcohol-based solvent or an ester-based solvent. After washing, the rinsing solution remaining on the substrate and the pattern may be removed. Also, in the case of using ultrapure water, water remaining on the substrate and the pattern may be removed.

In addition, the developer may be used alone or any combination of two or more different developers may also be used.

After the resist pattern is formed as described above, a pattered wiring substrate may be obtained by etching. Any etching methods well known in the art such as dry etching using plasma gas, and wet etching using an alkaline solution, a copper (II) chloride solution, or a ferric chloride solution may be used.

After forming the resist pattern, plating may be performed. Although a plating method is not particularly limited, for example, copper plating, solder plating, nickel plating, and gold plating may be used.

The resist pattern remaining after etching may be stripped off using an organic solvent. Examples of the organic solvent may be, but are not limited to, propylene glycol monomethylether acetate (PGMEA), propylene glycol monomethylether (PGME), and ethyl lactate (EL). Although a peeling method is not particularly limited, for example, immersing and spraying may be used. In addition, a wiring substrate on which the resist pattern is formed may be a multi-layered wiring substrate or may have a small-diameter through hole.

In an embodiment, the wiring substrate may be formed by a method including forming a resist pattern, depositing a metal thereon in a vacuum, and melting the resist pattern using a solution, i.e., a lift-off method.

Hereinafter, the disclosure will be described in more detail according to the following examples and comparative examples. However, the following examples are merely presented as example embodiments of the disclosure, and the scope of inventive concepts of the disclosure is not limited thereto.

### EXAMPLES

### Synthesis Example 1: Synthesis of P1

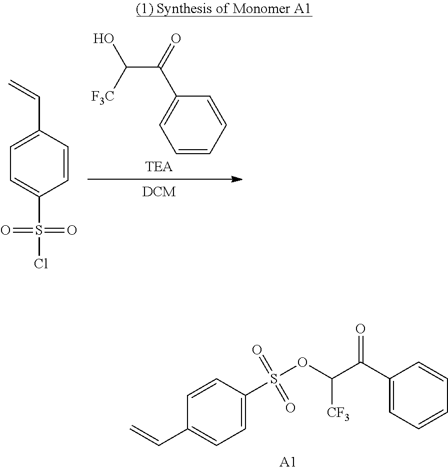 [SMILES: C=Cc1ccc(S(=O)(=O)Cl)cc1 ; O=C(c1ccccc1)C(O)C(F)(F)F ; C=Cc1ccc(S(=O)(=O)OC(C(=O)c2ccccc2)C(F)(F)F)cc1 ; "A1"]

2-hydroxy-2-trifluoromethylacetophenone (10 g, 49 mmol) was mixed with dichloromethane (DCM) (200 ml) and tetraethylamine (TEA) (7.5 ml, 53.9 mmol), and 4-vinylbenzenesulfonyl chloride (7.53 g, 37.2 mmol) was added thereto, followed by stirring at room temperature for 18 hours. Subsequently, an organic layer obtained by extraction using 200 ml of water and 200 ml of DCM was rinsed with a saturated NH₄Cl aqueous solution and a saturated NaCl aqueous solution and dried with MgSO₄, followed by filtration. A filtrate obtained therefrom was depressurized, and a residue obtained therefrom was separated and purified by silica gel column chromatography. A solid obtained therefrom was dissolved in ethylacetate (EA) (5 ml) and n-hexane (n-Hex) (15 ml) and placed in a refrigerator, and then a recrystallized product was filtered to obtain Monomer A1. ¹H-NMR date of the produced compound was identified.

¹H-NMR (500 MHZ, DMSO-d₆) δ 8.14 (d, J=8.2 Hz, 2H), 7.94 (d, J=8.5 Hz, 2H), 7.75 (t, J=7.2 Hz, 3H), 7.59 (t, J=7.1 Hz, 2H), 7.10 (q, J=6.6 Hz, 1H), 6.84 (dd, J=17.7, 11.0 Hz, 1H), 6.08 (d, J=17.7 Hz, 1H), 5.52 (d, J=11.0 Hz, 1H).

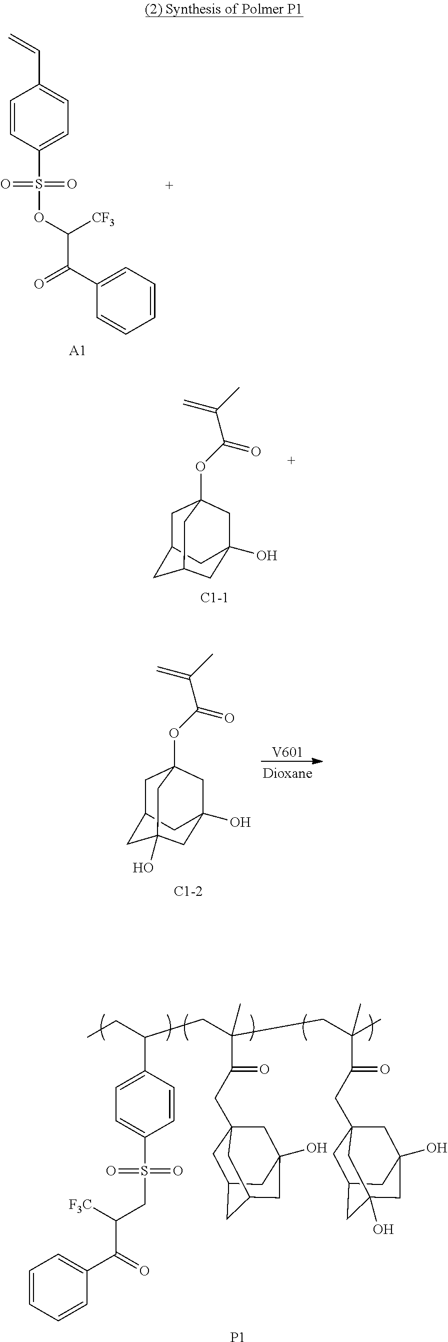

Monomer A1 (0.60 g, 1.6 mmol), Monomer C1-1 (0.31 g, 1.3 mmol), and Monomer C1-2 (0.082 g, 0.32 mmol), and V601 (0.15 g, 0.65 mmol), as an initiator, were dissolved in 1,3-dioxane (6.4 g), as a solvent. After stirring at 70° C. for 4 hours, white solid powder obtained by precipitation in n-hexane was filtered to obtain Polymer P1 (0.80 g, yield: 81%).

### Synthesis Examples 2 to 6

Polymers P2 to P6 were synthesized using the same method as that used to synthesize Polymer P1 according to Synthesis Example 1, except that monomers shown in Table 1 below were used in molar ratios of Table 1 instead of Monomer A1, Monomer C1-1, and Monomer C1-2. In this regard, all acetal groups contained in Monomer C1-3 used for synthesis of Polymers P5 and P6 were deprotected and converted into hydroxyl groups (phenol groups) during a polymerization process.

TABLE 1

| | | Input ratio | Weight average | |
|---|---|---|---|---|
| | | (molar | molecular | |
| Polymer | Monomer | ratio) | weight (Mw) | PDI |
| P1 | A1/C1-1/C1-2 | 50/40/10 | 6000 | 1.92 |
| P2 | A1/C1-1 | 50/50 | 6300 | 1.92 |
| P3 | A1/C1-2 | 50/50 | 6100 | 1.99 |
| P4 | A1/C2-1 | 50/50 | 7800 | 2.69 |
| P5 | A1/C1-3/C1-2 | 30/50/20 | 9500 | 2.22 |
| P6 | A1/C1-3/C2-1 | 20/60/20 | 6200 | 2.01 |

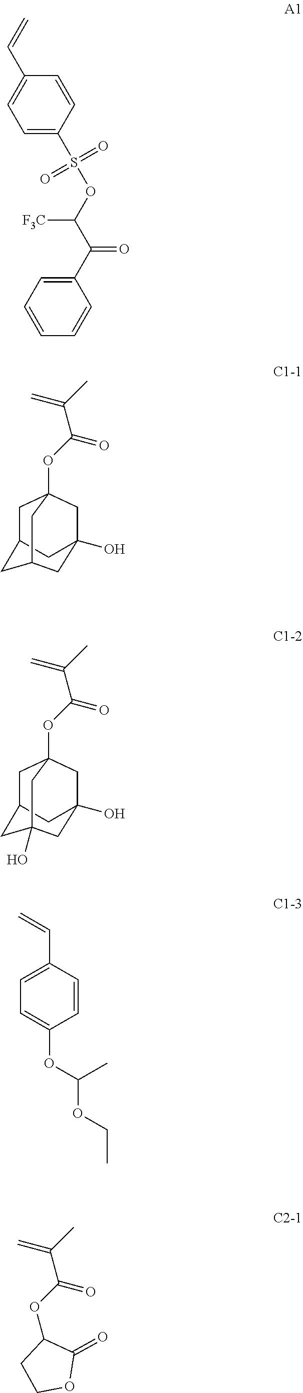

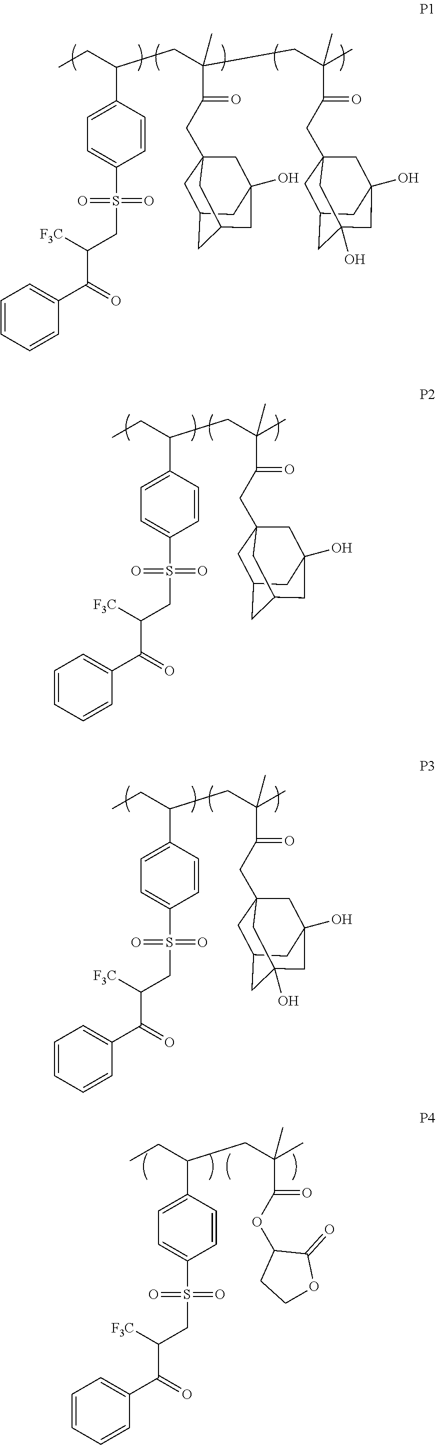

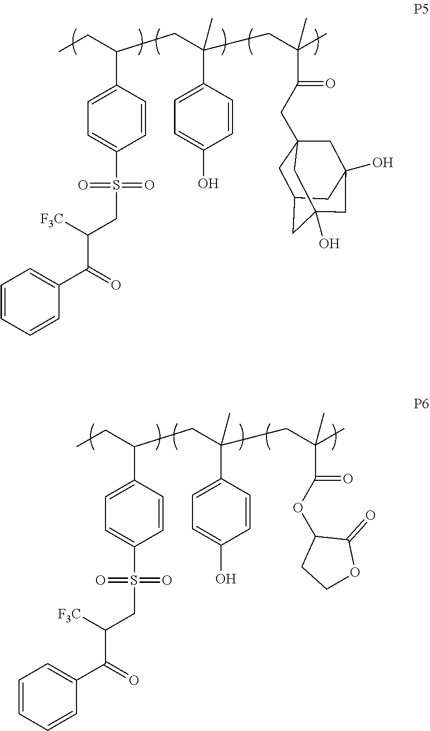

### Evaluation Example 1: Evaluation of Thin Film Development

### (1) Terms

E₀ refers to an exposure dose at a point where a thin film is completely developed (a thickness of the thin film is no longer decreased), and E₁ refers to an exposure dose at a point where development of the thin film is initiated. γ, indicating a contrast curve, is a value calculated by Equation 1 below.

$$γ = {[ log ( \frac{{E}_{0}}{{E}_{1}} ) ]}^{- 1} \qquad < Equation 1 >$$

NRT is an abbreviation for normalized remaining thickness.

### (2) Evaluation of Thin Film Development-Evaluation of Thin Film Development by DUV

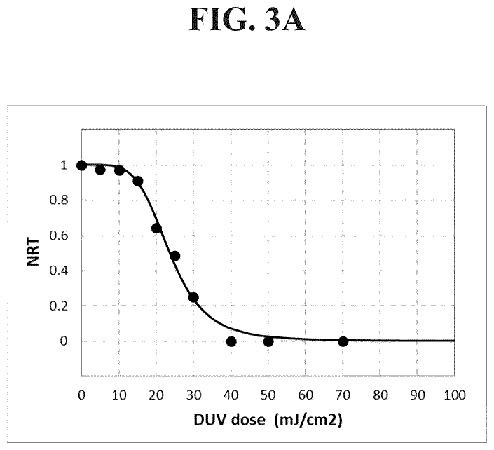

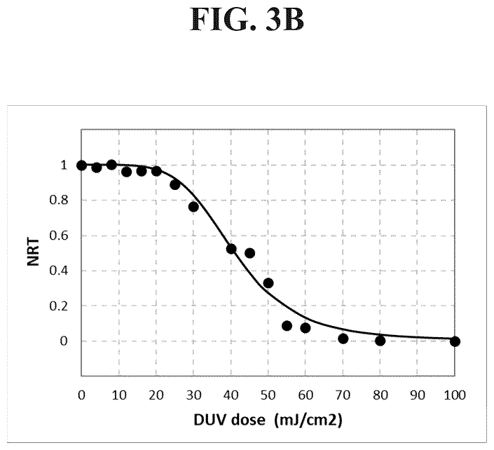

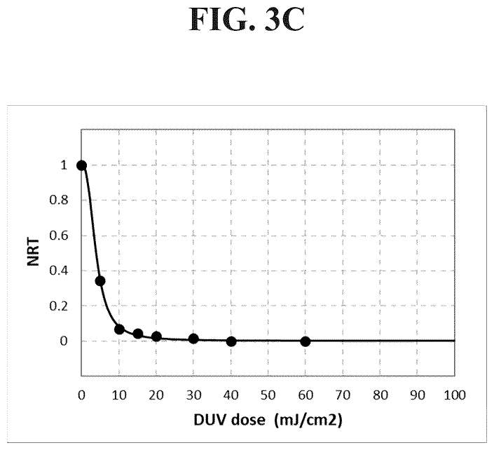

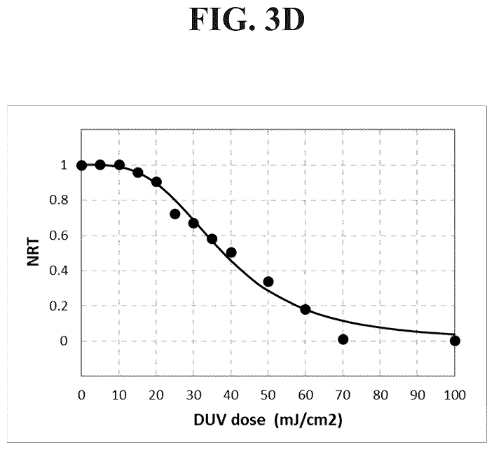

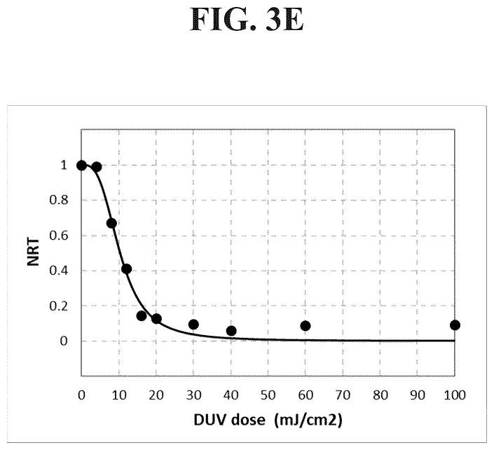

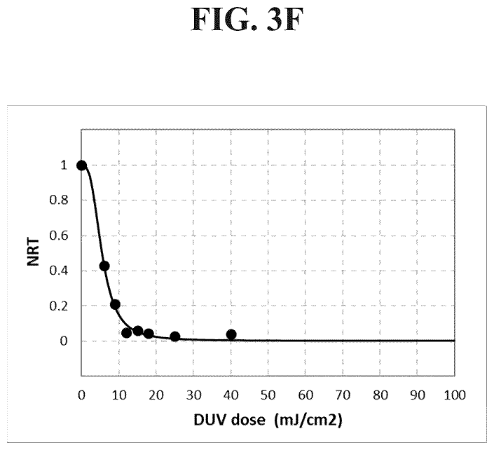

The polymers synthesized in Synthesis Examples 1 to 6 were dissolved in casting solvents of Table 2 shown below to 1.5 wt % to obtain a casting solution. An HMDS-treated silicon wafer was spin-coated with the casting solution at a speed of 1500 rpm and dried at 120° C. for 1 minute (PAB) to form a thin film having a thickness of 40 nm. Subsequently, the thin film was exposed to light having a wavelength of 254 nm (DUV) at a dose of 0 to 100 mJ/cm², and a film obtained after the light exposure was immersed in a developer of Table 2 at 25° C. for 60 seconds, washed with water, and naturally dried, and then a thickness of the remaining film was measured using a film thickness measurement instrument (Filmetrics®, F-20) and shown in Table 3 and FIGS. 3A to 3F. FIG. 3A is a graph showing data of Polymer 1 P1, FIG. 3B is a graph showing data of Polymer P2, FIG. 3C is a graph showing data of Polymer P3, FIG. 3D is a graph showing data of Polymer P4, FIG. 3E is a graph showing data of Polymer P5, and FIG. 3F is a graph showing data of Polymer P6.

TABLE 2

| Polymer | Casting solvent | Developer |
|---|---|---|
| P1 | PGME/PGMEA = 7/3(w/w) | 2.38 wt % TMAH aqueous |
| | | solution |
| P2 | PGME/PGMEA = 7/3(w/w) | 2.38 wt % TMAH aqueous |
| | | solution |
| P3 | PGME/PGMEA = 7/3(w/w) | 2.38 wt % TMAH aqueous |
| | | solution |
| P4 | PGME/PGMEA = 7/3(w/w) | 2.38 wt % TMAH aqueous |
| | | solution |
| P5 | PGME/PGMEA = 7/3(w/w) | 2.38 wt % TMAH aqueous |
| | | solution |
| P6 | PGME/PGMEA = 7/3(w/w) | 2.38 wt % TMAH aqueous |
| | | solution |

TABLE 3

| Polymer | E₁ | E₀ | γ |
|---|---|---|---|
| P1 | 15.7 | 35.6 | 2.81 |
| P2 | 27.5 | 61.3 | 2.87 |
| P3 | 1.8 | 8.5 | 1.47 |
| P4 | 20.8 | 69.4 | 1.91 |
| P5 | 5.4 | 19.8 | 1.76 |
| P6 | 2.7 | 10.9 | 1.67 |

Referring to Table 3 and FIGS. 3A to 3F, it was confirmed that solubility of all of the synthesized polymers in the developer changed after exposure to DUV rays. Also, it was confirmed that all of the synthesized polymers had high sensitivity to changes in solubility to the developer even at a low dose of 30 mJ/cm² or less.

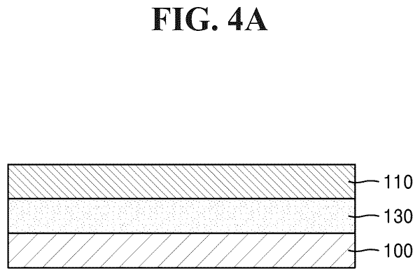

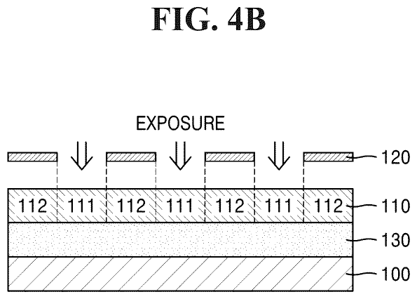

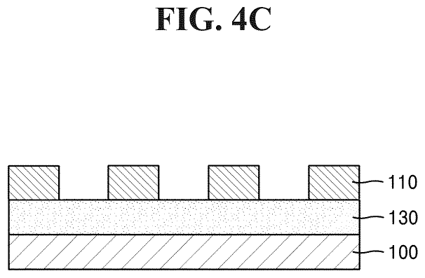

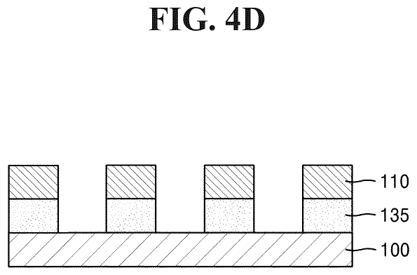

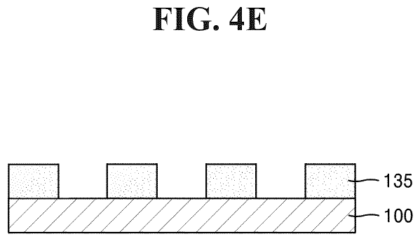

FIGS. 4A to 4E are side cross-sectional views illustrating a method of forming a patterned structure according to an embodiment.

Referring to FIG. 4A, a material layer 130 may be formed on the substrate 100 before forming a resist film 110 on the substrate 100. The resist film 110 may be formed on top of the material layer 130. The material layer 130 may include an insulating material (e.g., silicon oxide, silicon nitride), a semiconductor material (e.g., silicon), a metal (e.g., copper). In some embodiments, the material layer 130 may be a multi-layer structure. A material of the material layer 130 may be different than a material of the substrate 100. The resist film 110 may include a resist composition according to example embodiments and may have a thickness of 10 nm to 300 nm or 30 nm to 200 nm.

Referring to FIG. 4B, the resist film 110 may undergo a pre-exposure bake process and may be exposed with high energy rays through a mask 120, after which the resist film 110 may include exposed regions 111 and unexposed regions 112.

Referring to FIG. 4C, the exposed resist film 110 may be developed using a developer (e.g., developing solution). The exposed regions 111 may be washed away by the developer, whereas the unexposed regions 112 may remain without being washed away by the developer. Alternatively, the unexposed region 112 may be washed away from the developer and the exposed region 111 may remain.

Referring to FIG. 4D, exposed areas of the material layer 130 may be etched using the resist pattern 110 as a mask to form a material pattern 135 on the substrate 100.

Referring to FIG. 4E, the resist pattern 110 may be removed.

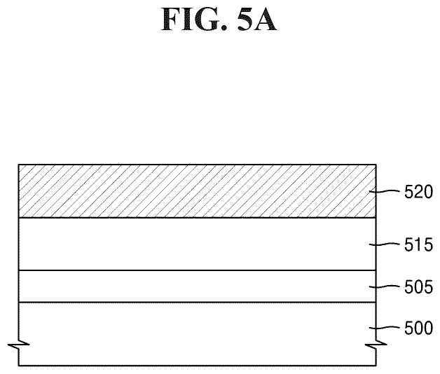

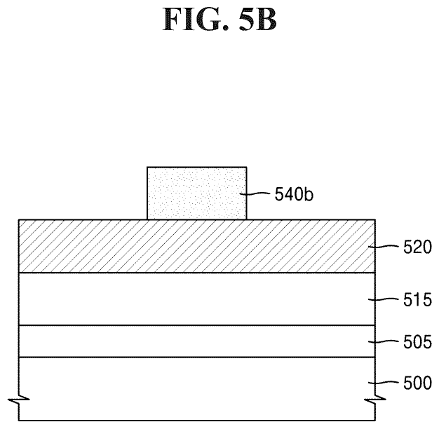

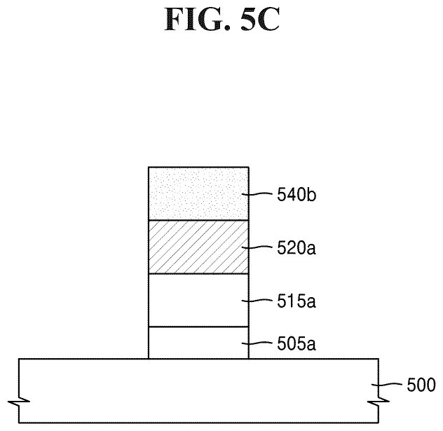

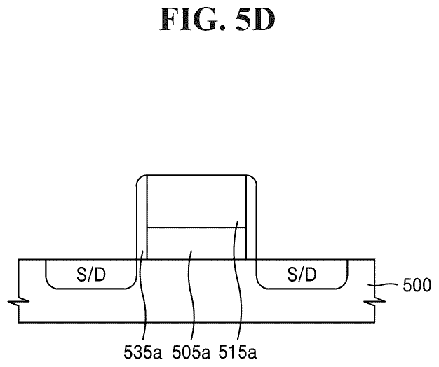

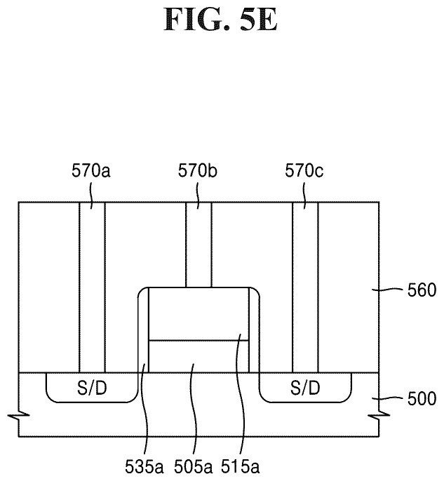

FIGS. 5A to 5E are side cross-sectional views illustrating a method of forming a semiconductor device according to an embodiment.

Referring to FIG. 5A, a gate dielectric 505 (e.g., silicon oxide) may be formed on a substrate 500. The substrate 500 may be a semiconductor substrate, such as a silicon substrate. A gate layer 515 (e.g., doped polysilicon) may be formed on the gate dielectric 505. A hardmask layer 520 may be formed on the gate layer 515.

Referring to FIG. 5B, a resist pattern 540b may be formed on the hardmask layer 520. The resist pattern 540b may be formed using a resist composition according to example embodiments. The resist pattern 540b may be formed from a resist composition including a photoacid generator, an organic solvent, and optionally a quencher and a base resin.

Referring to FIG. 5C, the gate layer 515 and the gate dielectric 505 may be etched to form a hardmask pattern 520a, a gate electrode pattern 515a, and a gate dielectric pattern 505a.

Referring to FIG. 5D, a spacer layer may be formed over the gate electrode pattern 515a and the gate dielectric pattern 505a. The spacer layer may be formed using a deposition process (e.g., CVD). The spacer layer may be etched to form spacers 535a (e.g., silicon nitride) on sidewalls of the gate electrode pattern 515a and the gate dielectric pattern 505a. After forming the spacers 535a, ions may be implanted into the substrate 500 to form source/drain impurity regions S/D.

Referring to FIG. 5E, an interlayer insulating layer 560 (e.g., oxide) may be formed on the substrate 500 to cover the gate electrode pattern 515a, gate dielectric pattern 505a, and spacers 535a. Then, electrical contacts 570a, 570b, and 570c may be formed in the interlayer insulating layer 560 to connect to the gate electrode 515a and the S/D regions. The electrical contacts may be formed of a conductive material (e.g., metal). Although not illustrated, a barrier layer may be formed between sidewalls of the interlayer insulating layer 560 and the electrical contacts 570a, 570b, and 570c. While FIGS. 5A to 5E illustrate an example of forming a transistor, inventive concepts are not limited thereto.

A resist composition according to one or more embodiments may be used in a patterning process to form other types of semiconductor devices.

According to embodiments of the present disclosure, provided are a polymer, a monomer, and a resist composition including the same, each having physical properties changing even at a low dose, and a method of forming a pattern using the same may be provided.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A polymer comprising:
a first repeating unit represented by Formula 1 below,
Formula 1
▪
wherein, in Formula 1,
L₁₁ to L₁₃ are each independently: a single bond; O; S; C(═O); C(═O)O; OC(═O); C(═O)NH; NHC(═O); or a linear, branched, or cyclic C₁-C₃₀ divalent hydrocarbon group optionally including a heteroatom,
a11 to a13 are each independently an integer from 1 to 4,
X₁₁ is a linear, branched, or cyclic C₁-C₃₀ divalent hydrocarbon group optionally including a heteroatom other than F,
Rf is a linear, branched, or cyclic C₁-C₃₀ monovalent hydrocarbon group including at least one F and optionally including a heteroatom other than F,
R₁₁ and R₁₂ are each independently: hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
R₁₃ is a substituted or unsubstituted C₆-C₃₀ aryl group or a substituted or unsubstituted C₁-C₃₀ heteroaryl group, and
an adjacent two among Rf, R₁₂, and R₁₃ are optionally bound to each other to form a ring, and
* is a binding site with an adjacent atom.

2. The polymer of claim 1, wherein the polymer does not comprise a repeating unit of which the structure is changed by an acid.

3. The polymer of claim 1, wherein X₁₁ is a linear, branched, or cyclic C₁-C₃₀ divalent hydrocarbon group optionally including O, S, N, or a combination thereof.

4. The polymer of claim 1, wherein Rf is a linear, branched, or cyclic C₁-C₃₀ monovalent hydrocarbon group including at least one F and optionally including O.

5. The polymer of claim 1, wherein Rf is includes one of a C₁-C₂₀ alkyl group substituted with at least one F, a C₁-C₂₀ alkoxy group substituted with at least one F, a C₃-C₂₀ cycloalkyl group substituted with at least one F, and a C₃-C₂₀ cycloalkoxy group substituted with at least one F.

6. The polymer of claim 1, wherein
R₁₁ and R₁₂ are each independently selected from:
hydrogen; deuterium; a halogen atom; a cyano group; a hydroxyl group; an amino group; a carboxylate group; a thiol group; and
a C₁-C₂₀ alkyl group, a C₃-C₂₀ cycloalkyl group, and a C₆-C₂₀ aryl group, each unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₅-C₂₀ cycloalkoxy group, a C₆-C₂₀ aryl group, or any combination thereof, and
R₁₃ is selected from a C₆-C₃₀ aryl group and a C₁-C₃₀ heteroaryl group, each unsubstituted or substituted with deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, a C₁-C₂₀ alkoxy group, a C₃-C₂₀ cycloalkyl group, a C₃-C₂₀ cycloalkoxy group, a C₆-C₂₀ aryl group, or any combination thereof.

7. The polymer of claim 1, wherein
the first repeating unit is represented by Formula 1-1 below,
<Formula 1-1>
▪
wherein in Formula 1-1,
L₁₁ to L₁₃, a11 to a13, X₁₁, Rf, and Rit to R₁₃ are as defined in Formula 1,
R₁₄ is hydrogen, deuterium, a halogen atom, a cyano group, a hydroxyl group, an amino group, a carboxylate group, a thiol group, an ester moiety, a sulfonate ester moiety, a carbonate moiety, a carbamate moiety, a lactone moiety, a sultone moiety, a carboxylic anhydride moiety, a C₁-C₂₀ alkyl group, a C₁-C₂₀ halogenated alkyl group, C₁-C₂₀ alkoxy group, a C₃-C₂₀ cycloalkyl group, a C₃-C₂₀ cycloalkoxy group, or a C₆-C₂₀ aryl group,
b14 is an integer from 1 to 5, and
* is a binding site with an adjacent atom.

8. The polymer of claim 1, wherein the first repeating unit is selected from compounds of Group I below:
<Group I>
▪.

9. The polymer of claim 1, further comprising:
a second repeating unit represented by Formula 2 below,
<Formula 2>
▪
wherein, in Formula 2,
L₂₁ to L₂₃ are each independently: a single bond; O; S; C(═O); C(═O)O; OC(═O); C(═O)NH; NHC(═O); or a linear, branched, or cyclic C₁-C₃₀ divalent hydrocarbon group optionally including a heteroatom,
a21 to a23 are each independently an integer from 1 to 4,
R₂₁ is: hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
X₂₁ is a non-acid labile group, and
* is a binding site with an adjacent atom.

10. The polymer of claim 9, wherein
X₂₁ is hydrogen; a halogen atom; a cyano group; a hydroxyl group; a carboxylate group; a thiol group; an amino group; or a linear, branched, or cyclic C₁-C₃₀ monovalent hydrocarbon group optionally including at least one polar moiety selected from a halogen atom, a cyano group, a hydroxyl group, a thiol group, a carboxylate group, O, C═O, C(═O)O, OC(═O), S(═O)O, OS(═O), a lactone moiety, a sultone moiety, and a carboxylic anhydride moiety.

11. The polymer of claim 9, wherein
X₂₁ is hydrogen, a hydroxyl group, or a group represented by one of Formulae 5-1 to 5-12 below:
▪
wherein, in Formulae 5-1 to 5-12,
a51 is 1 or 2;
R₅₁ to R₅₆ are each independently: a binding site with an adjacent atom; hydrogen; a hydroxyl group; a carboxylate group; an ester moiety; a carbonate moiety; a carbamate moiety; a lactone moiety; a carboxylic anhydride moiety; or a linear, branched, or cyclic C₁-C₂₀ monovalent hydrocarbon group,
one of R₅₁(s), R₅₂(s) and R₅₃, one of R₅₄(s), and one of R₅₅(s) and R₅₆(s) are binding sites with an adjacent atom,
b51 is selected from integers of 1 to 4,
b52 is selected from integers of 1 to 10,
b53 is selected from integers of 1 to 8,
b54 is selected from integers of 1 to 5,
b55 is selected from integers of 1 to 7,
b56 is selected from integers of 1 to 11,
b57 is selected from integers of 1 to 13,
b58 is selected from integers of 1 to 15,
b59 is selected from integers of 1 and 2, and
m51 is selected from integers of 1 to 4.

12. The polymer of claim 9, wherein
the second repeating unit is represented by Formula 2-1,
<Formula 2-1>
▪
wherein in Formula 2-1,
L₂₂ to L₂₃, a22 to a23, R₂₁, and X₂₁ are as defined in Formula 2,
L₂₄ is: a single bond; O; S; C(═O); C(═O)O; OC(═O); C(═O)NH; NHC(═O); or a linear, branched, or cyclic C₁-C₃₀ divalent hydrocarbon group optionally including a heteroatom,
a24 is an integer from 1 to 3, and
* is a binding site with an adjacent atom.

13. The polymer of claim 9, wherein
the second repeating unit is represented by one or more formulae selected from Group II below:
<Group II>
▪
▪
▪.

14. A resist composition comprising:
the polymer of claim 1; and
an organic solvent.

15. The resist composition of claim 14, wherein the resist composition does not comprise a photoacid generator.

16. The resist composition of claim 14, wherein the resist composition does not comprise a compound having a molecular weight of 1,000 or more in addition to the polymer.

17. A method of forming a pattern, the method comprising:
forming a resist film by coating the resist composition of claim 14 to a substrate;
exposing at least one portion of the resist film to high-energy rays to provide an exposed resist film; and
developing the exposed resist film using a developer.

18. The method of claim 17, wherein the exposing is performed by using at least one of ultraviolet (UV) rays, deep ultraviolet (DUV) rays, extreme ultraviolet (EUV) rays, X-rays, γ-rays, electron beams (EBs), or α-rays.

19. The method of claim 17, wherein
the exposed resist film has an exposed portion and an unexposed portion, and
the exposed portion is removed during the developing.

20. A monomer represented by Formula 10 below:
Formula 10
▪
wherein, in Formula 10,
Y₁₁ is a polymerizable group;
L₁₁ is: a single bond; O; S; C(═O); C(═O)O; OC(═O); C(═O)NH; NHC(═O); or a linear, branched, or cyclic C₁-C₃₀ divalent hydrocarbon group optionally including a heteroatom,
a11 is an integer from 1 to 4,
X₁₁ is a linear, branched, or cyclic C₁-C₃₀ divalent hydrocarbon group optionally including a heteroatom other than F,
Rf is a linear, branched, or cyclic C₁-C₃₀ monovalent hydrocarbon group including at least one F and optionally including a heteroatom other than F,
R₁₂ is: hydrogen; deuterium; a halogen atom; a cyano group; a hydroxyl group; an amino group; a carboxylate group; a thiol group; an ester moiety; a sulfonate ester moiety; a carbonate moiety; a carbamate moiety; a lactone moiety, a sultone moiety; a carboxylic anhydride moiety; or a linear, branched, or cyclic C₁-C₃₀ monovalent hydrocarbon group optionally including a heteroatom,
R₁₃ is a substituted or unsubstituted C₆-C₃₀ aryl group or a substituted or unsubstituted C₁-C₃₀ heteroaryl group, and
an adjacent two among Rf, R₁₂, and R₁₃ are optionally bound to each other to form a ring.
